(19) United States
(12) Patent Application Publication
(10) Pub. No.: US 2006/0182706 A1
(43) Pub. Date: Aug. 17, 2006

(54) Polymer products and their use in cosmetic preparations

(75) Inventors: Klemens Mathauer, Heidelberg (DE); Christian Drohmann, Brasschaat (DE); Marianna Pierobon, Ludwigshafen (DE); Claudia Wood, Weinheim (DE)

Correspondence Address: CONNOLLY BOVE LODGE & HUTZ LLP, SUITE 800, 1990 M STREET NW, WASHINGTON, DC, 20036-3425, US

(73) Assignee: BASF AKTIENGESELLSCAFT Patents, Trademarkds and Licenses, Ludwigshafen (DE)

(21) Appl. No.: 10/548,613

(22) PCT Filed: Feb. 20, 2004
(86) PCT No.: PCT/EP04/01672

§ 371 Date: Sep. 7, 2005

(30) Foreign Application Priority Data
Mar. 7, 2003 (DE) 103 10 378.3
Mar. 12, 2003 (DE) 103 11 120.4

(51) Int. Cl. A61K 8/81

(52) U.S. Cl. 424/70.16; 525/112

(57) ABSTRACT

The present invention relates to polymeric products obtainable by mixing (A) polymers which are obtainable by free-radical polymerization of (A-1) ethylenically unsaturated monomers in the presence of (A-2) unsaturated polyalkylene glycols with (B) silicones at a temperature greater than or equal to 30° C. In addition, the invention relates to a process for the preparation of these polymeric products, and to their use in cosmetic preparations, in particular in hair cosmetic preparations.

The present invention relates to polymeric products, to a process for their preparation, and to the use of the polymeric products in cosmetic, in particular hair cosmetic, preparations.

Polymers with form-forming properties are used for cosmetic and/or pharmaceutical preparations and are suitable in particular as additives for hair and skin cosmetics.

In cosmetic preparations for the skin, polymers can develop a particular effect. The polymers can, inter alia, contribute to the moisture retention and conditioning of the skin and to an improvement in the feel of the skin. The skin becomes smoother and more supple.

In cosmetic preparations for the hair, polymers are used for setting, improving the structure of and shaping the hair. They increase the combability and improve the feel of the hair. These hair-treatment compositions generally comprise a solution of the film former in an alcohol or a mixture of alcohol and water.

One requirement of hair-treatment compositions is to give the hair, inter alia, shine, flexibility and a natural, pleasant feel. The desired profile of properties includes strong hold at high atmospheric humidity, elasticity, ability to be washed out of the hair and compatibility with the other formulation components.

DE 42 40 108 describes polysiloxane-containing binders which are suitable as soil repellent coatings, in particular as antigraffiti coatings. However, these binders are paint-like and are not suitable for cosmetic purposes.

DE 16 45 569 describes a process for the preparation of organosilicon graft copolymers and their use as foam masses.

EP 0 953 015 describes amphiphilic polymers in combination with alkoxylated silicones.

JP 06-192048 describes copolymers of (meth)acrylamide-based monomers in combination with polyoxyalkylated siloxanes.

JP 10-226627 describes amphoteric polymers in combination with polyoxyalkylated siloxanes.

EP 0 852 488 B1 describes cosmetic compositions which comprise a silicone-containing graft polymer in combination with at least one further silicone.

It is an object of the present invention to provide polymeric products which are suitable in particular for hair cosmetic preparations. Of particular importance was the combination of different advantageous properties, such as strong hold at high atmospheric humidity, elasticity, ability to be washed out of the hair and compatibility with the other formulation components. Furthermore, the product should give the hair shine, flexibility and a natural, pleasant feel.

We have found that this object is achieved by polymeric products which are obtainable by mixing
(A) polymers which are obtainable by free-radical polymerization of (A-1) ethylenically unsaturated monomers in the presence of (A-2) unsaturated polyalkylene glycols with
(B) silicones
at a temperature greater than or equal to 30° C.

In a particularly preferred embodiment, the silicones (B) used are compounds which are chosen from the group consisting of (B-1) silicones with at least one quaternized or nonquaternized amine function, (B-2) silicone resins, (B-3) silicone rubbers, (B-4) polyalkoxylated silicones and/or (B-5) silicone-containing polyurethanes (B-5).

All of the indices and substituents given below refer in each case to the formulae which come directly before them in the text.

Ethylenically Unsaturated Monomers (A-1)

The suitable polymerizable monomers (A-1) used are ethylenically unsaturated monomers. In this connection, it is possible to use either individual monomers or combinations of two or more monomers.

Monomers which may be polymerized by a reaction initiated by free radicals are preferred. The term ethylenically unsaturated means that the monomers have at least one polymerizable carbon-carbon double bond, which may be mono-, di-, tri- or tetrasubstituted.

Polymerizable means that the monomers used can be polymerized using any conventional synthetic method.

The ethylenically unsaturated monomers (A-1) can be described by the following formula A-1a:
X—C(O)CR⁷═CHR⁶ (A-1a)
where

X is chosen from the group of radicals —OH, —OM, —OR⁸, NH₂, —NHR⁸, N(R⁸)₂;

M is a cation chosen from the group consisting of: Na+, K+, Mg++, Ca++, Zn++, NH₄+, alkylammonium, dialkylammonium, trialkylammonium and tetraalkylammonium;

the radicals R⁸ may be identical or different and are chosen from the group consisting of —H, C1-C40 linear or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.

R⁷ and R⁶, independently of one another, are chosen from the group consisting of: —H, C₁-C₈ linear or branched-chain alkyl chains, methoxy, ethoxy, 2-hydroxyethoxy, 2-methoxyethoxy and 2-ethoxyethyl.

Representative but nonlimiting examples of suitable monomers (A-1) are, for example, acrylic acid and salts, esters and amides thereof. The salts may be derived from any desired nontoxic metal, ammonium or substituted ammonium counterions.

The esters can be derived from C₁-C₄₀ linear, C₃-C₄₀ branched-chain, or C₃-C₄₀ carbocyclic alcohols, from polyfunctional alcohols having 2 to about 8 hydroxyl groups, such as ethylene glycol, hexylene glycol, glycerol and 1,2,6-hexanetriol, from aminoalcohols or from alcohol ethers, such as methoxyethanol and ethoxyethanol, or polyethylene glycols.

Also suitable are N,N-dialkylaminoalkyl acrylates and methacrylates and N-dialkylaminoalkylacrylamides and -methacrylamides of the formula A-1b 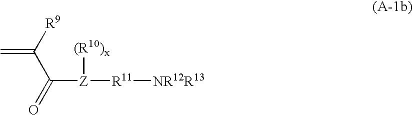
where R⁹=H, alkyl having 1 to 8 carbon atoms,
R¹⁰=H, methyl,
R¹¹=alkylene having 1 to 34 carbon atoms, optionally substituted by alkyl,
R¹², R¹³=C₁-C₄₀ alkyl radical,
Z=nitrogen when x=1 or oxygen when x=0.

The amides may be unsubstituted, N-alkyl- or N-alkylamino-monosubstituted, or N,N-dialkyl-substituted or N,N-dialkylamino-disubstituted, where the alkyl or alkylamino groups are derived from C₁-C₄₀ linear, C₃-C₄₀ branched-chain or C₃-C₄₀ carbocyclic units. In addition, the alkylamino groups may be quaternized.

Preferred monomers of the formula A-1b are N,N-dimethylaminomethyl (meth)acrylate, N,N-diethylaminomethyl (meth)acrylate, N,N-dimethylaminoethyl (meth)acrylate, N,N-diethylaminoethyl (meth)acrylate.

Monomers (A-1) which can likewise be used are substituted acrylic acids, and salts, esters and amides thereof, where the substituents on the carbon atoms are in the two or three position of the acrylic acid, and, independently of one another, are chosen from the group consisting of C₁-C₄ alkyl, —CN, COOH, particularly preferably methacrylic acid, ethacrylic acid and 3-cyanoacrylic acid. These salts, esters and amides of these substituted acrylic acids may be chosen as described above for the salts, esters and amides of acrylic acid.

Other suitable monomers (A-1) are vinyl and allyl esters of C₁-C₄₀ linear, C₃-C₄₀ branched-chain or C₃-C₄₀ carbocyclic carboxylic acids (e.g.: vinyl acetate, vinyl propionate, vinyl neononanoate, vinylneoundecanoic acid or vinyl t-butylbenzoate); vinyl or allyl halides, preferably vinyl chloride and ally chloride, vinyl ethers, preferably methyl, ethyl, butyl or dodecyl vinyl ethers, vinylformamide, vinylmethylacetamide, vinylamine; vinyl lactams, preferably vinylpyrrolidone and vinylcaprolactam, vinyl- or allyl-substituted heterocyclic compounds, preferably vinylpyridine, vinyloxazoline and allylpyridine.

Also suitable are N-vinylimidazoles of the formula A-1c, in which R¹⁴ to R¹⁶, independently of one another, are hydrogen,

C₁-C₄-alkyl or phenyl:

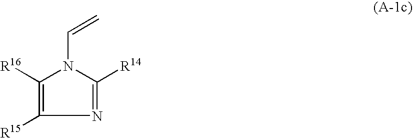

Further suitable monomers (A-1) are diallylamines of the formula A-1d 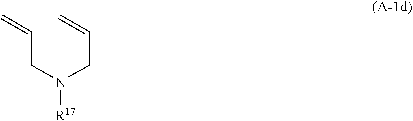
where R¹⁷═C₁-C₂₄ alkyl

Further suitable monomers (A-1) are vinylidene chloride; and hydrocarbons with at least one carbon-carbon double bond, preferably styrene, alpha-methylstyrene, tert-butylstyrene, butadiene, isoprene, cyclohexadiene, ethylene, propylene, 1-butene, 2-butene, isobutylene, vinyltoluene, and mixtures of these monomers.

Particularly suitable monomers (A-1) are acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids, such as, for example, acrylamidopropanesulfonic acid;

acrylamide, methacrylamide, ethacrylamide, N-methylacrylamide, N,N-dimethylacrylamide, N-ethylacrylamide, N-isopropylacrylamide, N-butylacrylamide, N-t-butylacrylamide, N-octylacrylamide, N-t-octylacrylamide, N-octadecylacrylamide, N-phenylacrylamide, N-methylmethacrylamide, N-ethylmethacrylamide, N-dodecylmethacrylamide, 1-vinylimidazole, 1-vinyl-2-methylimidazole, N,N-dimethylaminomethyl (meth)acrylate, N,N-diethylaminomethyl (meth)acrylate, N,N-dimethylaminoethyl (meth)acrylate, N,N-diethylaminoethyl (meth)acrylate, N,N-dimethylaminobutyl (meth)acrylate, N,N-diethylaminobutyl (meth)acrylate, N,N-dimethylaminohexyl (meth)acrylate, N,N-dimethylaminooctyl (meth)acrylate, N,N-dimethylaminododecyl (meth)acrylate, N-[3-(dimethylamino)propyl]methacrylamide, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]methacrylamide, N-[3-(diethylamino)propyl]methacrylamide, N-[3-(diethylamino)propyl]acrylamide;

maleic acid, fumaric acid, maleic anhydride and its monoesters, crotonic acid, itaconic acid, diallyldimethylammonium chloride, vinyl ethers (for example: methyl, ethyl, butyl or dodecyl vinyl ethers), vinylformamide, vinylmethylacetamide, vinylamine; methyl vinyl ketone, maleimide, vinylpyridine, vinylimidazole, vinylfuran, styrene, styrenesulfonate, allyl alcohol, and mixtures thereof.

Of these, particular prefrence is given to acrylic acid, methacrylic acid, maleic acid, fumaric acid, crotonic acid, maleic anhydride, and its monoesters, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, N-t-butylacrylamide, N-octylacrylamide, 2-hydroxyethyl acrylate, hydroxypropyl acrylate, 2-hydroxyethyl methacrylate, hydroxypropyl methacrylate, alkylene glycol (meth)acrylates, unsaturated sulfonic acids, such as, for example, acrylamidopropanesulfonic acid, vinylpyrrolidone, vinylcaprolactam, vinyl ethers (e.g.: methyl, ethyl, butyl or dodecyl vinyl ethers), vinylformamide, vinylmethylacetamide, vinylamine, 1-vinylimidazole, 1-vinyl-2-methylimidazole, N,N-dimethylaminomethyl methacrylate and N-[3-(dimethylamino)propyl]methacrylamide; 3-methyl-1-vinylimidazolium chloride, 3-methyl-1-vinylimidazolium methylsulfate, N,N-dimethylaminoethyl methacrylate, N-[3-(dimethylamino)propyl]methacrylamide quaternized with methyl chloride, methyl sulfate or diethyl sulfate.

In a very particularly preferred embodiment, the monomers (A-1) used are t-butyl acrylate and methacrylic acid.

Monomers with a basic nitrogen atom can be quaternized in the following way:

Suitable for quaternizing the amines are, for example, alkyl halides having 1 to 24 carbon atoms in the alkyl group, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic amines can also be carried out with alkylene oxides, such as ethylene oxide or propylene oxide, in the presence of acids. Preferred quaternizing agents are: methyl chloride, dimethyl sulfate or diethyl sulfate.

In a preferred embodiment, the monomers (A-1) used are (meth)acrylates.

The quaternization can be carried out before the polymerization or after the polymerization.

In addition, the reaction products of unsaturated acids, such as, for example, acrylic acid or methacrylic acid, with a quaternized epichlorohydrin of the formula A-1e (R¹⁸=C₁ to C₄₀ alkyl) can be used.

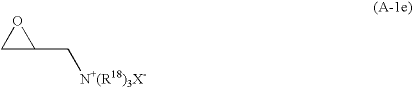

Examples thereof are, for example: (meth)acryloyloxyhydroxypropyltrimethylammonium chloride and (meth)acryloyloxyhydroxypropyltriethylammonium chloride.

The basic monomers can also be cationized by neutralizing them with mineral acids, such as, for example, sulfuric acid, hydrochloric acid, hydrobromic acid, hydroiodic acid, phosphoric acid or nitric acid, or with organic acids, such as, for example, formic acid, acetic acid, lactic acid, or citric acid.

In addition to the abovementioned monomers, the monomers (A-1) used may be so-called macromonomers, such as, for example, silicone-containing macromonomers with one or more free-radically polymerizable groups or alkyloxazoline macromonomers, as are described, for example, in EP 408 311.

Furthermore, it is possible to use fluorine-containing monomers, as are described, for example, in EP 558 423, compounds with a crosslinking action or compounds which regulate molecular weight, in combination or on their own.

Unsaturated Polyalkylene glycols (A-2)

Suitable as compounds (A-2) are unsaturated polyalkylene glycols of the formula
H₂C═CH—C_{c}H_{2c}—O—(C₂H₄O)ₐ(C₃H₆O)_{b}—R⁵ (A-2a)
and/or
H₂C═CH—C_{c}H_{2c}—O—(C₄H₈O)ₐ—R⁵ (A-2b)
in which

R⁵ is —H, —CH═CH₂, —(CH₂)ₛ—CH═CH—(CH₂)ₜ—H, a straight-chain or branched alkyl group having 1 to 12 carbon atoms, a straight-chain or branched alkoxy group having 1 to 6 carbon atoms, a straight-chain or branched acyl group having 2 to 40 carbon atoms, —SO₃M, a C₁₋₆-aminoacyl group optionally substituted on the amino group, —NHCH₂CH₂COOM, —N(CH₂CH₂COOM)₂, an aminoalkyl group optionally substituted on the amino group and on the alkyl group, a C₂₋₃₀-carboxyacyl group, a phosphono group optionally substituted by one or two substituted aminoalkyl groups, —CO(CH₂)_{d}COOM, —COCHR⁷(CH₂)_{d}COOM, —NHCO(CH₂)_{d}OH, —NH₂Y or a phosphate group
the groups M, which are identical or different from one another, are hydrogen, Na, K, Li, NH₄ or an organic amine,
R⁷ is hydrogen or SO₃M,
d is in the range from 1 to 10,
s may be 0, 1 to 10
t maybe0, 1 to 10
a is in the range from 0 to 50,
b is in the range from 0 to 50,
a+b is greater than 0,
c is 0, 1, 2, 3 or 4,
Y is a monovalent inorganic or organic anion.

Particular preference is given to compounds (A-2) where R⁵═H and/or a straight-chain or branched alkyl group having 1 to 12 carbon atoms, in particular —CH₃ and —C₂H₅.

Particular preference is given to compounds (A-2) where c=1.

Very particular preference is given to compounds (A-2) where R⁵═H and R⁵═—CH₃.

Very particular preference is given to compounds (A-2) where R⁵═H and c=1

Very particular preference is given to compounds (A-2) where R⁵═—CH₃ and c=1

Compounds (A-2) are available commercially, for example under the trade names Pluriol from BASF AG. Examples which may be mentioned are the products Pluriol® A 10 R, Pluriol® A 11 R.

Likewise suitable are the compounds which are obtainable under the CAS No. 272 74-31-3 polyalkylene glycol monoallyl ether or under the CAS No. 126682-74-4 as polyalkylene glycol monovinyl ether from Clariant (description of A-and V-type polyglycols from Clariant).

Silicones (B)

Suitable silicones (B) are in principle all silicone compounds. Nonvolatile silicones are particularly suitable. Nonvolatile silicones are those compounds with a boiling point above 90° C., in particular above 100° C. Water-soluble or water-dispersible silicones are particularly suitable.

For the purposes of the invention, water-dispersible silicones means silicones which, upon contact with water, within 24 hours form a fluid which does not allow any solid particles to be detected with the eye without optical devices. To check whether a silicone is water-dispersible, 100 mg of the silicone, in the form of a 100 mm thick film, are placed in 100 ml of water (20° C.) and shaken for 24 hours on a commercially available shaking table. If, after shaking, solid particles can no longer be seen, but the fluid possesses turbidity, the silicone is water-dispersible; in the absence of turbidity, it is referred to as water-soluble.

In a preferred embodiment, the silicones used are compounds which are chosen from the group consisting of (B-1) silicones with at least one quaternized or nonquaternized amine function, (B-2) silicone resins, (B-3) silicone rubbers, (B-4) polyalkoxylated silicones and/or (B-5) silicone-containing polyurethanes. It is of course possible to use either mixtures of compounds within an individual group (e.g. 2 compounds from B-4) or else mixtures of compounds from different groups (one compound from B-1 and one or more compounds from, for example, B-4).

(B-1) Silicones with at Least one Quaternized or Nonquaternized Amine Function

Among the silicones which comprise at least one quaternized or nonquaternized amino group, mention may be made of:

(a) the silicone polymers which correspond to the formula B-1a:
R¹ₐG¹₃₋ₐ—Si(OSiG²₂)ₙ-(OSiG³_{b}R²_{2-b})ₘ—O—SiG⁴₃₋ₐ-R³ₐ (B-1a)
in which:

G¹, G², G³ and G⁴, which are identical or different, are a hydrogen atom, a phenyl group, OH, C₁-C₁₈-alkyl, for example methyl, C₂-C₁₈-alkenyl or C₁-C₁₈-alkoxy;

a, a′, which are identical or different, are 0 or an integer from 1 to 3, in particular 0;

b is 0 or 1 and in particular 1;

m and n are numbers whose sum (n+m) can vary particularly between 1 and 2000 and in particular 50 and 150, where n can be a number from 0 to 1999 and particularly from 49 to 149 and m can be a number from 1 to 2000, particularly from 1 10;

R¹, R², R³, R⁴, which are identical or different, are a monovalent radical of the formula C_{q}H_{2q}OₛR⁵ₜL, in which q is a number from 1 to 8, s and t, which are identical or different, are 0 or 1, R⁵ is an optionally hydroxylated alkylene group and L is an optionally quaternized aminated group which is chosen from the following groups:
NR″—CH₂—CH₂—N′(R″)₂
N(R″)₂
N′(R″)₃A⁻
N′H(R″)₂A⁻
N′H₂(R″)A⁻
N(R″)—CH₂—CH₂—N′R″H₂A⁻,
   in which R″ is hydrogen, phenyl, benzyl or a monovalent saturated hydrocarbon radical, e.g. an alkyl radical having 1 to 20 carbon atoms, and A⁻ is a halide ion, such as, for example, fluoride, chloride, bromide or iodide.

(b) the compounds of the formula B-1b:
NH—[(CH₂)₃—Si[OSi(CH₃)₃]]₃ (B-1b)

This compound corresponds to the CTFA name “Amino-bis-propyldimethicone”. The products corresponding to formula B-1a are, for example, the polysiloxanes, which are referred to in CTFA nomenclature as “Amodimethicones” and correspond to the following formula B-1b: 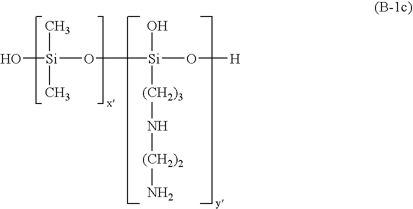
in which x′ and y′ are integers which depend on the molecular weight and are generally those whose molecular weight is between 5000 and approximately 20 000. A product which corresponds to the formula B-1a is the polymer named in CTFA nomenclature as “Trimethylsilylamodimethicone”, which corresponds to the formula B-1d: 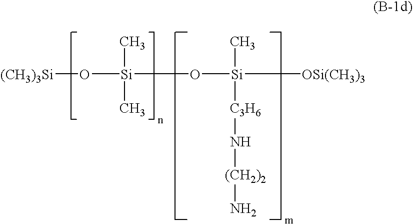
in which n and m have the meanings given above (cf. formula B-1a). A commercial product which corresponds to this definition is a mixture (90/10% by weight of a polydimethylsiloxane with aminoethyl-aminoisobutyl groups and a polydimethylsiloxane which is sold under the name Q2-8220 by DOW CORNING. Such polymers are described, for example, in the patent application EP-A-95238.

Other polymers which correspond to the formula B-1a are silicone polymers of the formula: 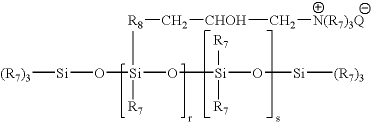
in which:
R⁷ is a monovalent hydrocarbon radical having 1 to 18 carbon atoms and in particular a C₁-C₁₈-alkyl radical or C₂-C₁₈-alkenyl radical, e.g. methyl;
R⁸ is a divalent hydrocarbon radical, particularly a C₁-C₁₈-alkylene radical or a divalent C₁-C₁₈-alkenoxy radical, e.g. C₁-C₈;
Q⁻ is a halide ion, in particular chloride;
r is a statistical average value from 2 to 20 and in particular from 2 to 8;
s is a statistical average value from 20 to 200 and in particular from 20 to 50.

Such polymers are described in particular in the U.S. Pat. No. 4,185,087.

A polymer which corresponds to the formula B 1b is the polymer which is sold by Union Carbide under the name “Ucar Silicone ALE 56”. If these silicone polymers are used, a particularly interesting embodiment is their use together with cationic and/or nonionic surfactants. For example, it is possible to use the product sold under the name “Emulsion Cationique DC 929” by DOW CORNING, which, apart from the amodimethicone, comprises a cationic surfactant which comprises a mixture of products according to the following formula: 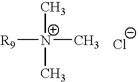
in which R⁹ is alkenyl and/or alkyl radicals having 14 to 22 carbon atoms, derivatives of tallow fatty acids, together with a nonionic surfactant of the formula: C₉H₁₉—C₆H₄—(OC₂H₄)₁₀—OH, known under the CTFA nomenclature “Nonoxynol 10”.

Another commercial product which can be used according to the invention is the product which is sold under the name “Dow Corning Q2 7224” by Dow Corning which, together with the trimethylsilylamodimethicone of the formula B-1d, comprises a nonionic surfactant of the formula: C₈H₁₇—C₆H₄—(OCH₂CH₂)ₙ—OH, in which n=40, otherwise referred to as octoxynol-40, a further nonionic surfactant of the formula: C₁₂H₂₅—(OCH₂—CH₂)ₙ—OH, in which n=6, otherwise referred to as isolaureth-6, and glycol.

(B-2) Silicone Resins

The silicon resins which can be used according to the invention are crosslinked siloxane systems which comprise the units: R²SiO_{2/2}, RSiO_{3/2}, and SiO_{4/2}, in which R is a hydrocarbon group which has 1 to 6 carbon atoms, or is a phenyl group. Among these products, particular preference is given to those in which R is a lower alkyl radical (C₁-C₆) or a phenyl radical.

Among these resins, mention may be made of the product which is sold under the name “DOW CORNING 593”, or any products which are sold under the names “SILICONE FLUID SS 4230” and “SS 4267” by GENERAL ELECTRIC and which are “dimethyl/trimethylpolysiloxanes”.

(B-3) Silicone Rubbers

The silicone rubbers (B-3) according to the invention are polydiorganosiloxanes of high molar masses between 200 000 and 2 000 000, which are used on their own or in a mixture in a solvent which is chosen from the volatile silicones, the polydimethylsiloxane oils, the polymethylphenylsiloxane or the polydiphenyldimethylsiloxane oils, the isoparaffins, methylene chloride, pentane, the hydrocarbons or their mixtures.

Preference is given to using a silicone rubber with a molecular weight below 1 500 000. The silicon rubbers are, for example, a polydimethylsiloxane, a polyphenylmethylsiloxane, a poly(diphenylsiloxan-dimethylsiloxane), a poly(dimethylsiloxane-methylvinylsiloxane), a poly(dimethylsiloxane-phenylmethylsiloxane), a poly(diphenylsiloxane-dimethylsiloxane-methylvinylsiloxane). These silicone rubbers can terminate at the end of the chain with trimethylsilyl or dimethylhydroxysilyl groups.

In particular, a silicone rubber may be used which corresponds to the formula B-3a: 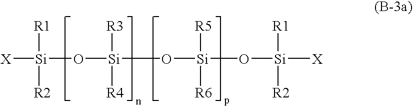
in which:
R¹, R², R⁵ and R⁶, together or separately, are an alkyl radical having 1 to 6 carbon atoms,
R³ and R⁴, together or separately, are an alkyl radical having 1 to 6 carbon atoms or an aryl radical,
X is an alkyl radical having 1 to 6 carbon atoms, a hydroxyl radical or a vinyl radical,
where n and p are chosen such that the silicone rubber has a viscosity greater than 100 000 mPa.s, preferably greater than 500 000 mPa.s.

In general, n and p can have values from 0 to 5000, preferably from 0 to 3000. The silicone rubber can be incorporated into the composition as it is or in a form dissolved in silicone oil, such as a volatile or nonvolatile PDMS (polydimethylsiloxane).

Silicone rubbers which can be used according to the invention may be any in which:
the substituents R¹ to R⁶ and X is a methyl group, p=0 and n=2700, such as that which is sold under the name SE30 by General Electric,
the substituents R¹ to R⁶ and X are a methyl group, p=0 and n=2300, such as that which is sold under the name AK 500 000 by Wacker,
the substituents R¹ to R⁶ are a methyl group, the substituent X is a hydroxyl group, p=0 and n=2700, in a 13% strength solution in cyclopentasiloxane, such as that which is sold under the name Q2-1401 by DOW CORNING,
the substituents R¹ to R⁶ are a methyl group, the substituent X is a hydroxyl group, p=0 and n=2700, in a 13% strength solution in dimethicone, such as that which is sold under the name Q2-1403 by DOW CORNING,
the substituents R₁, R₂, R₅, R₆ and X are a methyl group, the substituents R³ and R⁴ are an aryl group, and the molecular weight of the compound is 600 000, such as that which is sold under the name 761 by RHONE-POULENC.
   (B4) Polyalkoxylated Silicones

Polyalkoxylated silicones (B4) are compounds which are chosen from the compounds of the formulae: 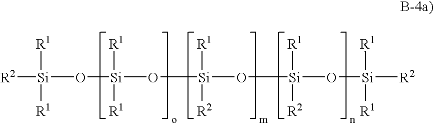 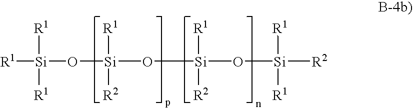 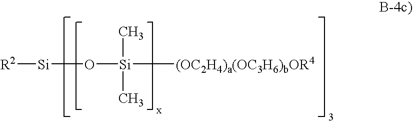 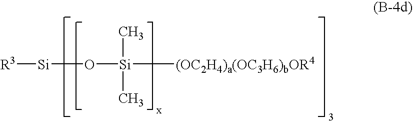
where, in the formulae B-4a, B-4b, B-4c and B-4d
the groups R¹, which are identical or different from one another, are a straight-chain or branched C₁ to C₃₀-alkyl group or a phenyl group, in particular —CH₃
the groups R², which are identical or different from one another, are R¹ or a group —C_{c}H_{2c}—O—(C₂H₄O)ₐ(C₃H₆O)_{b}—R⁵ or a group C_{c}H_{2c}—O—(C₄H₈O)ₐ—R⁵, with the proviso that at least one of the radicals R² is a group
—C_{c}H_{2c}—O—(C₂H₄O)ₐ(C₃H₆O)_{b}—R⁵ or a group C_{c}H_{2c}—O—(C₄H₈O)ₐ—R⁵,
the groups R³ and R⁴, which are identical or different from one another, are a straight-chain or branched C₁ to C₁₂-alkyl group and preferably methyl,
the groups R⁵, which are identical or different from one another, are chosen from a hydrogen atom, a straight-chain or branched alkyl group having 1 to 12 carbon atoms, a straight-chain or branched alkoxy group having 1 to 6 carbon atoms, a straight-chain or branched acyl group having 2 to 40 carbon atoms, —SO₃M, a C₁₋₆-aminoalkoxy group optionally substituted on the amino group, a C₂₋₆-aminoacyl group optionally substituted on the amino group, —NHCH₂CH₂COOM, —N(CH₂CH₂COOM)₂, an aminoalkyl group optionally substituted on the amino group and on the alkyl group, a C₂₋₃₀-carboxyacyl group, a phosphono group optionally substituted by one or two substituted aminoalkyl groups, —CO(CH₂)_{d}COOM, —COCHR₇(CH₂)_{d}COOM, —NHCO(CH₂)_{d}OH, —NH₃Y or a phosphate group,
the groups M, which are identical or different from one another, are hydrogen, Na, K, Li, NH₄ or an organic amine,
R₇ is hydrogen or SO₃M,
d is in the range from 1 to 10,
m is in the range from 0 to 20,
n is in the range from 0 to 500,
o is in the range from 0 to 20,
p is in the range from 1 to 50,
a is in the range from 0 to 50,
b is in the range from 0 to 50,
a+b is at least 2,
c is in the range from 0 to 4,
x is in the range from 1 to 100, and
Y is a monovalent inorganic or organic anion,
with the proviso that n is greater than 12 if the silicone corresponds to the formula B-4b where R₅=hydrogen.

The polyalkoxylated silicones according to the invention can likewise be chosen from the silicones of the following formula B-4e:
([Z(R₂SiO)_{q}R′₂SiZO][(CₙH₂ₙO)ᵣ])₅ (B-4e)
in which:
R₂ and R′₂, which are identical or different, are a monovalent hydrocarbon-containing radical,
n is an integer from 2 to 4,
q is a number of at least 4, preferably between 4 and 200 and in particular between 4 and 100,
r is a number of at least 4, preferably between 4 and 200 and in particular between 5 and 100,
s is a number of at least 4, preferably between 4 and 1000 and in particular between 5 and 300,
Z is a divalent organic group which is bonded via a carbon-silicon bond to the adjacent silicon atom and via an oxygen atom to a polyoxyalkylene block,
the average molecular weight of each siloxane block is between 400 and about 10 000, that of each polyoxyalkylene block is between about 300 and about 10 000,
the siloxane blocks are about 10 to about 95% by weight of the block copolymer,
the number-average molecular weight of the block copolymer can be between 2500 to 1 000 000 and preferably between 3000 ad 200 000 and in particular between 6000 and 100 000.

R₂ and R′₂ are preferably chosen from the group which comprises the straight-chain or branched alkyl radicals, such as, for example, the methyl, ethyl, propyl, butyl, pentyl, hexyl, octyl, decyl, dodecyl radicals, the aryl radicals, such as, for example, the phenyl, naphthyl radicals, the aralkyl radicals or alkylaryl radicals, such as, for example, the benzyl radicals, the phenylethyl radicals, the tolyl radicals and the xylyl radicals.

Z is preferably —R″—, —R″—CO—, —R″—NHCO—, —R″—NH—CO—NH—R″—, —R′″—OCONH—R′″—NHCO—, where R″ is a divalent, straight-chain or branched alkylene group having 1 to 6 carbon atoms, such as, for example, ethylene, propylene or butylene, straight-chain or branched, and R′″ is a divalent alkylene group or a divalent arylene group such as —C₆H₄—, —C₆H₄—C₆H₄—, —C₆H₄CH₂—C₆H₄—, —C₆H₄C(CH₃)₂C₆H₄—.

In a more preferred manner, Z is a divalent alkylene radical, in particular the radical —C₃H₆ or the radical C₄H₈, straight-chain or branched.

The preparation of the block copolymers used for the purposes of this invention is described in European application EP 0 492 657 A1, the teaching of which is included in this description.

Such products are marketed, for example, under the name SILICONFLUID FZ-2172 by OSI.

The silicones according to the invention can be in the form of aqueous solutions or optionally in the form of aqueous dispersions or emulsions.

Silicone-Containing Polyurethanes (B-5)

Further silicones which can be used are silicone-containing polyurethanes. Of particular suitability are water-soluble or water-disppersible polyurethanes derived from
a) at least one polysiloxane,
b) at least one polyesterdiol,
c) at least one compound with a molecular weight in the range from 56 to 300, which comprises two active hydrogen atoms per molecule,
d) at least one compound which has two active hydrogen atoms and at least one anionogenic or anionic group per molecule,
e) at least one diisocyanate,
   or the salts thereof, where the polyurethane comprises no unit which originates from a primary or secondary amine which has an ionogenic or ionic group.

Component a) is preferably a polymer with a number-average molecular weight in the range from about 400 to 4000, preferably 500 to 4000, in particular 600 to 3000.

The polysiloxanes a) are preferably a compound of the formula B-5a 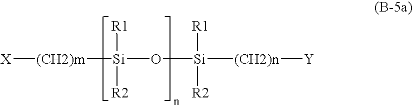
I
in which
R¹ and R², independently of one another, are C₁- to C₄-alkyl, benzyl or phenyl,
X and Y, independently of one another, are OH or NHR³, where R³ is hydrogen, C₁- to C₆-alkyl or C₅- to C₈-cycloalkyl,
m and n, independently of one another, are 2 to 8,
p is 3 to 50.

Suitable alkyl radicals are, for example, methyl, ethyl, n-propyl, isopropyl, n-butyl, t-butyl, n-pentyl, n-hexyl etc. Suitable cycloalkyl radicals are, for example, cyclopentyl, cyclohexyl, cycloheptyl, cyclooctyl etc.

Preferably, R¹ and R² are both methyl.

Polyesterdiols b) which can be used have a number-average molecular weight in the range from about 400 to 5000, preferably 500 to 3000, in particular 600 to 2000.

Suitable polyesterdiols are all those which are usually used for the preparation of polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, and poly(meth)acrylate-diols of the formula B-5b 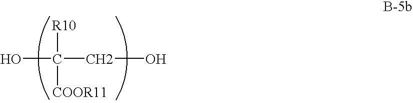
in which R¹⁰ is H or CH₃ and R¹¹ is C₁-C₁₈-alkyl (in particular C₁-C₁₂- or C₁-C₈-alkyl), which have a molar mass of up to about 3000. Such diols can be prepared in the usual manner and are commercially available (Tegomer™ grades MD, BD and OD from Goldschmidt).

Preference is given to polyesterdiols based on aromatic and aliphatic dicarboxylic acids and aliphatic diols, in particular those in which the aromatic dicarboxylic acid constitutes 10 to 95 mol %, in particular 40 to 90 mol % and preferably 50 to 85 mol %, of the total dicarboxylic acid fraction (remainder is aliphatic dicarboxylic acids).

Particularly preferred polyesterdiols are the reaction products of phthalic acid/diethylene glycol, isophthalic acid/1,4-butanediol, isophthalic acid/adipic acid/1,6-hexanediol, 5-NaSO₃-isophthalic acid/phthalic acid/adipic acid/1,6-hexanediol, adipic acid/ethylene glycol, isophthalic acid/adipic acid/neopentyl glycol, isophthalic acid/adipic acid/neopentyl glycol/diethylene glycol/dimethylolcyclohexane and 5-NaSO₃-isophthalic acid/isophthalic acid/adipic acid/neopentyl glycol/diethylene glycol/dimethylolcyclohexane.

Component c) is preferably diols, diamines, aminoalcohols and mixtures thereof. The molecular weight of these compounds is preferably in a range from about 56 to 280. If desired, up to 3 mol % of said compounds may be replaced by triols or triamines. The resulting polyurethanes here are essentially uncrosslinked.

As component c), preference is given to using diols. Diols which can be used are, for example, ethylene glycol, propylene glycol, butylene glycol, neopentyl glycol, dimethylolcyclohexane, di-, tri-, tetra-, penta- or hexaethylene glycol and mixtures thereof. Preference is given to using neopentyl glycol and/or dimethylolcyclohexane.

Suitable aminoalcohols are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc.

Suitable diamines are, for example, ethylenediamine, propylenediamine, 1,4-diaminobutane, 1,5-diaminopentane and 1,6-diaminohexane, and α,ω-diaminopolyethers which can be prepared by amination of polyalkylene oxides with ammonia.

Suitable compounds d) which have two active hydrogen atoms and at least one ionogenic or anionic group per molecule are, for example, compounds with carboxylate and/or sulfonate groups. As component d), particular preference is given to dimethylolpropanoic acid and mixtures which comprise dimethylolpropanoic acid.

As component d), it is also possible to use compounds of the formulae
H₂N(CH₂)ₙ—NH—(CH₂)ₘ—COO⁻M⁺
H₂N(CH₂)ₙ—NH—(CH₂)ₘ—SO₃⁻M⁺
in which m and n, independently of one another, are an integer from 1 to 8, in particular 1 to 6, and M is Li, Na or K. As component d), preference is given to using mixtures which have dimethylolpropanoic acid and up to 3% by weight, based on the total amount of components a) to e), of at least one compound of the abovmentioned formulae.

Component e) is customary aliphatic, cycloaliphatic and/or aromatic diisocyanates, such as tetramethylene diisocyanate, hexamethylene diisocyanate, methylenediphenyl diisocyanate, 2,4- and 2,6-tolylene diisocyanate and isomeric mixtures thereof, 1,5-naphthylene diisocyanate, 1,4-cyclohexylene diisocyanate, dicyclohexylmethane diisocyanate and mixtures thereof, in particular isophorone diisocyanate and/or dicyclohexylmethane diisocyanate. If desired, up to 3 mol % of said compounds may be replaced by triisocyanates.

Preferably, the silicon-containing polyurethanes (B-5) used are compounds derived from
0.5 to 40% by weight, preferably 2 to 30% by weight, of at least one polysiloxane a),
1 to 45% by weight, preferably 2 to 35% by weight, of at least one polyester-diol b),
0.3 to 15% by weight, preferably 0.5 to 12% by weight, of at least one component c),
5 to 25% by weight, preferably 8 to 20% by weight, of at least one component d),
25 to 60% by weight, preferably 35 to 53% by weight, of at least one component e).

Preferably, this is a silicon-containing polyurethane derived from
0.2 to 20% by weight, preferably 0.5 to 15% by weight, in particular 1 to 10% by weight, of at least one polysiloxane a),
10 to 45% by weight, preferably 15 to 40% by weight, of at least one polyester-diol b),
0.3 to 15% by weight, preferably 0.5 to 12% by weight, of at least one component c),
5 to 25% by weight, preferably 8 to 20% by weight, of at least one component d),
25 to 60% by weight, preferably 35 to 53% by weight, of at least one component e).

The silicone-containing polyurethanes are prepared by reacting the compounds of components a), b), c) and d) with the component e). The temperature here is in a range from about 60 to 140° C., preferably about 70 to 100° C. The reaction can be carried out without solvents or in a suitable inert solvent or solvent mixture. Suitable solvents are aprotic-polar solvents, e.g. tetrahydrofuran, ethyl acetate, N-methylpyrrolidone, di-methylformamide and preferably ketones, such as acetone and methyl ethyl ketone. Preferably, the reaction is carried out under an inert gas atmosphere, such as, for example, under nitrogen. The components are used in amounts such that the ratio of NCO equivalent of the compounds of component e) to equivalent of active hydrogen atom of components a), b), c) and d) is in a range from about 0.8:1 to 1.25:1, preferably 0.85:1 to 1.2:1, in particular 1.05:1 to 1.15:1. If the resulting polyurethanes still have free isocyanate groups, these are subsequently deactivated by adding amines, preferably aminoalcohols. Suitable aminoalcohols are those described above as component c), preferably 2-amino-2-methyl-1-propanol.

The polyurethanes comprising acid groups can be converted into a water-soluble or water-dispersible form by partial or complete neutralization with a base.

As a rule, the resulting salts of the polyurethanes have better water solubility or dispersibility in water than the nonneutralized polyurethanes. Bases which can be used for the neutralization of the polyurethanes may be alkali metal bases, such as sodium hydroxide solution, potassium hydroxide solution, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and ammonia and amines. Suitable amines are, for example, C₁-C₆-alkylamines, preferably n-propylamine and n-butylamine, dialkylamines, preferably diethylpropylamine and dipropylmethylamine, trialkylamines, preferably triethylamine and triisopropylamine, C₁-C₆-alkyldiethanolamines, preferably methyl- or ethyldiethanolamine and di-C₁-C₆-alkylethanolamines. For use in hair-treatment compositions in particular, 2-amino-2-methyl-1-propanol, diethylaminopropylamine and triisopropanolamine have proven successful for the neutralization of the polyurethanes comprising acid groups. The neutralization of the polyurethanes comprising acid groups can also be undertaken using mixtures of two or more bases, e.g. mixtures of sodium hydroxide and triisopropanolamine. The neutralization can be carried out partially, e.g. to 20 to 40%, or completely, i.e. to 100%, depending on the intended use.

If, in the preparation of the polyurethanes, a water-miscible organic solvent is used, then this can be removed subsequently by customary methods known to the person skilled in the art, e.g. by distillation at reduced pressure. Before separating off the solvent, water can additionally be added to the polyurethane. Replacing the solvent with water gives a solution or dispersion of the polymer from which, if desired, the polymer can be obtained in the customary manner, e.g. by spray drying.

The silicone-containing polyurethanes (B-5) have K values (measured in accordance with E. Fikentscher, Cellulose-Chemie 13 (1932), pp. 58-64, on a 1% strength solution in N-methylpyrrolidone) in a range from 15 to 90, preferably 20 to 60. Their glass transition temperature is generally at least 0° C., preferably at least 20° C., especially preferably at least 25° C. and specifically at least 30° C.

If the silicone-containing polyurethanes (B-5) used are polyurethanes with units derived from polysiloxanes of the formula B-5a, then the proportion of siloxane groups, based on the solids content of the silicone-containing polyurethanes, is generally about 0.05 to 20% by weight, preferably about 0.05 to 15% by weight, in particular 0.05 to 10% by weight.

Particularly preferred silicones are polyalkoxylated silicones (B-4).

The present invention further provides a process for the preparation of polymeric products, which comprises
i) polymerizing ethylenically unsaturated monomers (A-1) in the presence of unsaturated polyalkylene glycol vinyl ethers (A-2)
ii) mixing the resulting polymer with silicones at a temperature greater than or equal to 30° C.
   i) Preparation of Polymers (A)

The polymers (A) can be prepared by customary conventional synthetic methods of polymerization. For example, these may be solution polymerization, emulsion polymerization, inverse emulsion polymerization, suspension polymerization, inverse suspension polymerization or precipitation polymerization, without the methods which can be used being limited thereto. In solution polymerization, water, customary organic solvents or the unsaturated polyalkylene glycol vinyl ethers (A-2) can themselves be used as solvents.

The regulators which may be used are the customary compounds known to the person skilled in the art, such as, for example, sulfur compounds (e.g.: mercaptoethanol, 2-ethylhexyl thioglycolate, thioglycolic acid or dodecyl mercaptan), and tribromochloromethane and other compounds which have a regulating effect on the molecular weight of the resulting polymers. It is also possible in some instances to use thiol group-containing silicone compounds. Preference is given to using silicone-free regulators.

In one embodiment of the invention, additional crosslinking monomers are used in the preparation of the polymers (A). Crosslinking monomers which may be used are compounds with at least two ethylenically unsaturated double bonds, such as, for example, esters of ethylenically unsaturated carboxylic acids, such as acrylic acid or methacrylic acid, and polyhydric alcohols, ethers of at least dihydric alcohols, such as, for example, vinyl ether or allyl ether. Also suitable are straight-chain or branched, linear or cyclic aliphatic or aromatic or aromatic hydrocarbons which have at least two double bonds, which in the case of the aliphatic hydrocarbons must not be conjugated. Also suitable are amides of acrylic acid and methacrylic acid and N-allylamines of at least difunctional amines, such as, for example, 1,2-diaminoethane, 1,3-diaminopropane. Also are triallylamine or corresponding ammonium salts, N-vinyl compounds of urea derivatives, at least difunctional amides, cyanurates or urethanes. Further suitable crosslinkers are divinyldioxane, tetraallylsilane or tetravinylsilane.

Particularly preferred crosslinkers are, for example, methylenebisacrylamide, triallylamine and triallylammonium salts, divinylimidazole, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

The monomers (A-1) according to the invention can, if they comprise ionizable groups, be neutralized partially or completely with acids or bases before or after the polymerization in order, for example, to adjust the solubility or dispersibility in water to a desired extent.

Neutralizing agents for monomers carrying acid groups which can be used are, for example, mineral bases, such as sodium carbonate, alkali metal hydroxides and ammonia, organic bases, such as aminoalcohols, specifically 2-amino-2-methyl-1-propanol, monoethanolamine, diethanolamine, triethanolamine, triisopropanolamine, tri[(2-hydroxy)-1-propyl]amine, 2-amino-2-methyl-1,3-propanediol, 2-amino-2-hydroxymethyl-1,3-propanediol, and diamines, such as, for example, lysine.

Neutralizing agents which can be used for monomers carrying cationizable groups may, for example, be mineral acids, such as hydrochloric acid, sulfuric acid or phosphoric acid, and organic acids, such as carboxylic acids, lactic acid, citric acid or others.

The monomers (A-1 ) of the polymers (A) can constitute from 50 to 99.9% by weight, preferably 70 to 99% by weight, particularly preferably 85 to 98% by weight, in particular 80 to 97% by weight.

The monomers (A-2, unsaturated polyalkylene glycol vinyl ethers) are generally present in the polymer (A) in amounts of from 0.1 to 50, preferably from 0.5 to 20, particularly preferably from 2 to 15, % by weight.

In a preferred embodiment of the invention, the monomers (A-1) used are at least 2 monomers (a1 and a2). Particular preference is given to polymers (A) which are obtainable by polymerization of monomer (a1) tert-butyl acrylate and monomer (a2) methacrylic acid. If the ethylenically unsaturated monomers (A-1) are used as a combination of two monomers (a1 and a2), it has proven advantageous to use 49.5 to 99% by weight of (a1) and 0.5 to 40% by weight of (a2).

In a particularly preferred embodiment, the polymer (A) used is a polymer which is obtainable by free-radical polymerization of a monomer mixture of
(a1) 49.5 to 99% by weight of (meth)acrylate, in particular tert-butyl acrylate
(a2) 0.5 to 40% by weight of a further (meth)acrylate, in particular methacrylic acid
(b) 0.5 to 20% by weight of an unsaturated polyalkylene glycol ether (A-2)

In a particularly preferred embodiment, the polymer (A) used is a polymer which is obtainable by free-radical polymerization of a monomer mixture of
(a1) 49.5 to 99% by weight of (meth)acrylate, in particular tert-butyl acrylate
(a2) 0.5 to 40% by weight of a further (meth)acrylate, in particular methacrylic acid
(c) 0.5 to 20% by weight of an unsaturated polyalkylene glycol ether (A-2) of the following formula
   H₂C═CH—C_{c}H_{2c}—O—(C₂H₄O)ₐ(C₃H₆O)_{b}—R⁵
where R⁵═H, CH₃,
a is in the range from 0 to 50,
b is in the range from 0 to 50,
a+b is greater than 0,
c is 0, 1, 2, 3 or 4.

The polymers (A) preferably have a K value (measured according to Fickentscher, Cellulosechemie, vol. 13, pp. 58-64 (1932) at 250° C. 0.1 5% strength in 0.5 molar sodium chloride solution) of from 30 to 50, preferably from 37 to 41.

Particularly suitable polymers (A) are those which are water-soluble or whose dispersibility in water is so great that they are soluble in a solvent mixture of water:ethanol=20:80 (% by volume:% by volume) in an amount of more than 0.1 g/l, preferably more than 0.2 g/l.

For the purposes of the invention, water-dispersible polymers means polymers which, upon contact with water, within 24 hours form a fluid which does not allow any solid particles to be detected by the eye without optical devices. To check whether a polymer is dispersible in water, 100 mg of the polymer in the form of a 100 mm thick film are placed in 100 ml of water (20° C.) and shaken for 24 hours on a commercially available shaking table. If, after the shaking, solid particles can no longer be seen, but the fluid possesses turbidity, the polymer is water-dispersible; without turbidity, it is referred to as water-soluble.

In the polymerization of the monomers (A-1) and (A-2), it is optionally possible for other polymers, such as, for example, homopolymers and copolymers of ethylenically unsaturated monomers, and also polyamides, polyurethanes or polyesters, to also be present. The polyamides, polyurethanes, polyesters are preferably ionically modified, e.g. with carboxylate or sulfonate groups.

ii) Mixing of Polymers (A) with Silicones (B)

The polymeric products according to the invention are obtainable in the simplest case by mixing the components (A) and (B). It is essential according to the invention that the temperature during mixing is greater than or equal to 30° C., in particular greater than or equal to 40° C.

In a particularly preferred embodiment of the invention, the polymeric products according to the invention are prepared by mixing (A) and (B) at temperatures greater than or equal to 50° C., in particular greater than or equal to 60° C., particularly preferably greater than or equal to 70° C.

It has proven advantageous to carry out the mixing for at least 30 minutes, in particular for at least 60 minutes.

For the mixing, all apparatuses known to a person skilled in the art are suitable. The mixing can be carried out with inert-gas gassing, e.g. nitrogen gassing.

In a preferred embodiment, 99.5 to 70% by weight, in particular 99 to 85% by weight, of (A) and 0.5 to 30% by weight, in particular 1 to 15%, by weight of (B), are used.

The polymeric products according to the invention are suitable in particular for use in cosmetic preparations, particularly in hair cosmetic preparations.

The present invention therefore further provides for the use of the polymeric products in cosmetic preparations.

For example, the polymeric products according to the invention are used in cosmetic compositions for cleansing the skin. Such cosmetic cleansing compositions are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, lubricating soaps and washing pastes, and liquid washing, showering and bathing preparations, such as washing lotions, shower baths and gels, foam baths, oil baths and scrub preparations.

Preferably, the polymeric products according to the invention are used in cosmetic compositions for the care and protection of the skin, in nailcare compositions, and in preparations for decorative cosmetics.

Particular preference is given to the use in skincare compositions, intimate care compositions, footcare compositions, deodorants, light protection compositions, repellents, shaving compositions, hair removal compositions, antiacne compositions, make-up, mascara, lipsticks, eyeshadows, kohl pencils, eyeliners, blushers, powders and eyebrow pencils.

The skincare compositions are, in particular, in the form of W/O or O/M skin creams, day creams and night creams, eye creams, face creams, antiwrinkle creams, moisturizing creams, bleaching creams, vitamin creams, skin lotions, care lotions and moisturizing lotions.

In the cosmetic preparations, the polymeric products according to the invention can develop particular effects. They can, inter alia, contribute to the moisturization and conditioning of the skin and to the improvement in the feel of the skin. By adding the polymeric products according to the invention, it is possible, in certain formulations, to achieve a considerable improvement in the skin compatibility.

The polymeric products according to the invention are present in the skin cosmetic preparations in an amount of from about 0.001 to 20% by weight, preferably from 0.01 to 10% by weight, very particularly preferably from 0.1 to 5% by weight, based on the total weight of the composition.

Depending on the field of use, the compositions according to the invention can be applied in a form suitable for skincare, such as, for example, in the form of a cream, foam, gel, pencil, powder, mousse, milk or lotion.

In addition to the dispersions according to the invention and suitable solvents, the skin cosmetic preparations may also comprise additives customary in cosmetics, such as emulsifiers, preservatives, perfume oils, cosmetic active ingredients, such as phytantriol, vitamin A, E and C, retinol, bisabolol, panthenol, light protection agents, bleaches, colorants, tinting agents, tanning agents (e.g. dihydroxyacetone), collagen, protein hydrolysates, stabilizers, pH regulators, dyes, salts, thickeners, gelling agents, consistency-imparting agents, silicones, humectants, refatting agents and further customary additives.

Suitabe solvents are, in particular, water and lower monoalcohols or polyols having 1 to 6 carbon atoms or mixtures thereof; preferred monoalcohols or polyols are ethanol, isopropanol, propylene glycol, glycerol and sorbitol.

Further customary additives which may be present are fatty substances, such as mineral and synthetic oils, such as, for example, paraffins, silicone oils and aliphatic hydrocarbons with more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, petroleum jelly, hydrogenated lanolin and acetylated lanolin. It is of course also possible to use mixtures thereof.

Customary thickeners in such formulations are crosslinked polyacrylic acids and derivatives thereof, polysaccharides, such as xanthan gum, agar agar, alginates or Tyloses, carboxymethylcellulose or hydroxycarboxymethylcellulose, fatty alcohols, monoglycerides and fatty acids, polyvinyl alcohol and polyvinylpyrrolidone.

The polymeric products according to the invention can be mixed with conventional polymers if specific properties are to be set.

Suitable conventional polymers are, for example, anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer™ 100P), copolymers of ethyl acrylate and methacrylic acid (e.g. Luvimer™ MAE), copolymers of N-tert-butylacrylamide, ethyl acrylate, acrylic acid (Ultrahold™ 8, strong), copolymers of vinyl acetate, crotonic acid and optionally further vinyl esters (e.g. Luviset™ grades), maleic anhydride copolymers, optionally reacted with alcohols, anionic polysiloxanes, e.g. carboxyfunctional ones, copolymers of vinylpyrrolidone, t-butyl acrylate, methacrylic acid (e.g Luviskol™ VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ethers and hyaluronic acid, Luviset P.U.R., Luviflex™ Silk.

Further suitable polymers are cationic polymers with the INCI name Polyquaternium, e.g. copolymers of vinylpyrrolidone/N-vinylimidazolium salts (Luviquat™ FC, Luviquat™ HM, Luviquat™ MS, Luviquat™ Care, Luviquat™ Hold, INCl Polyquaternium-16, -44, -46), copolymers of acrylamide and dimethyldiallylammonium chloride (Polyquaternium-7), cationic cellulose derivatives (Polyquaternium-4, -10), cationic starch derivatives (INCl: Starch Hydroxypropytrimonium Chloride, Corn Starch Modified), cationic guar derivatives (INCl: Hydroxypropyl Guar Hydroxypropyltrimonium Chloride), cationic sunflower oil derivatives (INCl: Sunflowerseedamidopropyl Hydroxyethyldimonium Chloride), copolymers of N-vinypyrrolidone/dimethylaminoethyl methacrylate, quaternized with diethyl sulfate (Polyquaternium-11), copolymers of acrylic acid, acrylamide and methacrylamidopropyltrimonium chloride (Polyquaternium-53), Polyquaternium-32, Polyquaternium-28 and others.

Suitable further polymers are also neutral polymers, such as polyvinylpyrrolidones, copolymers of N-vinylpyrrolidone and vinyl acetate and/or vinyl propionate, copolymers of N-vinypyrrolidone/dimethylaminopropylacrylamide or -methacrylamide, copolymers of N-vinylpyrrolidone and alkyl acrylate or methacrylate monomers with alkyl chains from C1 to C18, graft copolymers of polyvinyl alcohol onto polyalkylene glycols, such as, for example, Kollicoat IR (BASF), graft copolymers of other vinyl monomers onto polyalkylene glycols, polysiloxanes, polyvinylcaprolactam and copolymers containing N-vinylpyrrolidone, polyethylenimines and salts thereof, polyvinylamines and salts thereof, cellulose derivatives, chitosan, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins, dimethicones, dimethicone derivatives or dimethicone copolyols (CTFA) and aminofunctional silicone compounds, such as amodimethicones (CTFA).

The polymeric products according to the invention are used in cosmetic preparations whose preparation takes place in accordance with the customary guidelines familiar to the person skilled in the art.

Such formulations are advantageously in the form of emulsions, preferably in the form of water-in-oil (W/O) or oil-in-water (O/W) emulsions. It is, however, also possible according to the invention and in some cases advantageous to choose other types of formulation, for example hydrodispersions, gels, oils, oleogels, multiple emulsions, for example in the form of W/O/W or O/W/O emulsions, anhydrous ointments or ointment bases etc.

The preparation of emulsions which can be used according to the invention takes place in accordance with known methods.

In addition to the polymeric products according to the invention, the emulsions comprise customary constituents, such as fatty alcohols, fatty acid esters and in particular fatty acid triglycerides, fatty acids, lanolin and derivatives thereof, natural or synthetic oils or waxes and emulsifiers in the presence of water.

The choice of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, which is hereby expressly incorporated by reference.

Thus, a skin cream which can be used according to the invention may, for example, be in the form of a W/O emulsion. Such an emulsion comprises an aqueous phase which is emulsified in an oil or fatty phase using a suitable emulsifier system.

The concentration of the emulsifier system in this type of emulsion is about 4 and 35% by weight, based on the total weight of the emulsion; the fatty phase constitutes about 20 and 60% by weight and the aqueous phases about 20 and 70% by weight, in each case based on the total weight of the emulsion. The emulsifiers are those which are customarily used in this type of emulsion. They are chosen, for example, from: C₁₂-Cl₁₈-sorbitan fatty acid esters; esters of hydroxystearic acid and C12-C₃₀-fatty alcohols; monoesters and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium, calcium, lithium, zinc or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils whose distillation start-point under atmospheric pressure is at about 250° C. and whose distillation end-point is at 410° C., such as, for example, vaseline oil; esters of saturated or unsaturated fatty acids, such as alkyl myristates, e.g. isopropyl, butyl or cetyl myristate, hexadecyl stearate, ethyl or isopropyl palmitate, octanoic or decanoic triglycerides and cetyl ricinoleate.

The fatty phase can also comprise silicone oils soluble in other oils, such as dimethylpolysiloxane, methylphenylpolysiloxane and the silicone glycol copolymer, fatty acids and fatty alcohols.

In order to promote the retention of oils, it is also possible to use waxes, such as, for example, carnauba wax, candellila wax, beeswax, microcrystalline wax, ozokerite wax and Ca, Mg and Al oleates, myristates, linoleates and stearates.

In general, these water-in-oil emulsions are prepared by introducing the fatty phase and the emulsifier into the batch container. This is heated at a temperature of from 70 to 75° C., then the oil-soluble ingredients are added and, with stirring, water which has been heated beforehand to the same temperature and in which the water-soluble ingredients have been dissolved beforehand is added; the mixture is stirred until an emulsion of the desired fineness is achieved, which is then left to cool to room temperature, if necessary with a lesser amount of stirring.

In addition, a care emulsion according to the invention can be in the form of an O/W emulsion. Such an emulsion usually comprises an oil phase, emulsifiers which stabilize the oil phase in the water phase, and an aqueous phase, which is usually present in thickened form.

The aqueous phase of the O/W emulsion of the preparations according to the invention optionally comprises
alcohols, diols or polyols, and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; and also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are preferably O/W emulsifiers, such as polyglycerol esters, sorbitan esters or partially esterified glycerides.

The preparation can also take place by melting the oil phase at about 80° C.; the water-soluble constituents are dissolved in hot water, and added to the oil phase slowly and with stirring; the mixture is homogenized and stirred until cold.

The polymeric products according to the invention are also suitable for use in washing and shower gel formulations, and bathing preparations.

As well as the polymeric products according to the invention, such formulations usually comprise anionic surfactants as base surfactants and amphoteric and nonionic surfactants as cosurfactants, and also lipids, perfume oils, dyes, organic acids, preservatives and antioxidants, and also thickeners/gel formers, skin conditioners and humectants.

In the washing, showering and bathing preparations, all anionic, neutral, amphoteric or cationic surfactants customarily used in body-cleansing compositions may be used.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, alkylglycol alkoxylates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, it is possible to use cocodimethylsulfopropylbetaine, laurylbetaine, cocamidopropylbetaine or sodium cocamphopropionate.

Suitable nonionic surfactants are, for example, the reaction products of aliphatic alcohols or alkylphenols having 6 to 20 carbon atoms in the alkyl chain, which may be linear or branched, containing ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

In addition, the washing, showering and bathing preparations can comprise customary cationic surfactants, such as, for example, quaternary ammonium compounds, for example cetyltrimethylammonium chloride or bromide (INCl Cetrimonium chloride or bromide), hydroxyethylcetyldimonium phosphate (INCl Quaternium-44), INCl Cocotrimonium methosulfate, INCl Quaternium-52.

In addition, further customary cationic polymers can also be used, such as, for example, copolymers of acrylamide and dimethyldiallylammonium chloride (Polyquaternium-7), cationic cellulose derivatives (polyquaternium-4, -10), cationic starch derivatives (INCl: Starch Hydroxypropytrimonium Chloride, Corn Starch Modified), cationic guar derivatives (INCl: Hydroxypropyl Guar Hydroxypropyltrimonium Chloride), cationic sunflower oil derivatives (INCl: Sunflowerseedamidopropyl Hydroxyethyldimonium Chloride), copolymers of N-vinylpyrrolidone and quaternized N-vinylimidazole (Polyquaternium-16, -44, -46), copolymers of N-vinypyrrolidone/dimethylaminoethyl methacrylate, quaternized with diethyl sulfate (Polyquaternium-11), copolymers of acrylic acid, acrylamide and methacrylamidopropyltrimonium chloride (Polyquaternium-53), Polyquaternium-32, Polyquaternium-28 and others.

In addition, the washing and shower gel formulations and bathing preparations can comprise thickeners, such as, for example, sodium chloride, PEG-55, propylene glycol oleate, PEG-120 methyl glucose dioleate and others, and also preservatives, further active ingredients and auxiliaries and water.

In a preferred embodiment of the invention, the polymeric products are used in hair cosmetic preparations.

Hair cosmetic preparations include, in particular, styling compositions and/or conditioning compositions in hair cosmetic preparations such as hair treatments, hair mousses, (hair) gels or hair sprays, hair lotions, hair rinses, hair shampoos, hair emulsions, split end fluids, neutralizing agents for permanent waves, hair colorants and bleaches, hot-oil treatment preparations, conditioners, setting lotions or hair sprays. Depending on the field of use, the hair cosmetic preparations can be applied in the form of an (aerosol) spray, (aerosol) mousse, gel, gel spray, cream, lotion or wax.

In a preferred embodiment, the hair cosmetic formulations according to the invention comprise

a) 0.05 to 20% by weight of the polymeric product

b) 20 to 99.95% by weight of water and/or alcohol

c) 0 to 79.5% by weight of further constituents

Alcohol is understood as meaning all alcohols customary in cosmetics, e.g. ethanol, isopropanol, n-propanol.

Further constituents are understood as meaning additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, emollients, effect substances, opacifiers, active ingredients, antioxidants, peroxide decomposers, UV filters, care substances such as panthenol, collagen, vitamins, protein hydrolysates, alpha- and beta-hydroxycarboxylic acids, protein hydrolysates, stabilizers, pH regulators, dyes, pigments, viscosity regulators, gel formers, salts, humectants, refatting agents, complexing agents and further customary additives.

These also include all styling and conditioning polymers known in cosmetics which may be used in combination with the polymers according to the invention if very particular properties are to be set.

Suitable conventional hair cosmetic polymers are, for example, anionic polymers. Such anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset™ P.U.R.) and polyureas. Particularly suitable polymers are copolymers of t-butyl acrylate, ethyl acrylate, methacrylic acid (e.g. Luvimer™ 100P), copolymers of N-tert-butylacrylamide, ethyl acrylate, acrylic acid (e.g. Ultrahold™ 8, Strong), copolymers of vinyl acetate, crotonic acid and optionally further vinyl esters (e.g. Luviset™ grades, INCl: VA/Crotonates Copolymer), maleic anhydride copolymers, optionally reacted with alcohols, anionic polysiloxanes, e.g. carboxyfunctional ones, copolymers of vinylpyrrolidone, t-butyl acrylate, methacrylic acid (e.g Luviskol™ VBM).

In addition, the group of polymers suitable for combination with the polymeric products according to the invention includes, for example, Balance CR or 0/55 (National Starch; acrylate copolymer), Balance 47 (National Starch; octylacrylamide/acrylate/butylaminoethyl methacrylate copolymer), Aquaflex™ FX 64 (ISP; isobutylene/ethylmaleimide/hydroxyethylmaleimide copolymer), Aquaflex™ SF-40 (ISP/National Starch; VP/vinyl caprolactam/DMAPA acrylat copolymer), Allianz™ LT-120 (ISP/Rohm & Haas; acrylate/C1-2 succinate/hydroxyacrylate copolymer), Aquarez™ HS (Eastman; polyester-1), Diaformer™ Z-400 (Clariant; methacryloylethylbetaine/methacrylate copolymer), Diaformer™ Z-711 or Z-712 (Clariant; methacryloylethyl N-oxide/methacrylate copolymer), Omnirez™ 2000 (ISP; monoethyl ester of poly(methyl vinyl ether/maleic acid in ethanol), Amphomer™ HC or Resyn XP or Resyn 28-4961 (National Starch; acrylate/octylacrylamide copolymer), Amphomer™ 28-4910 (National Starch; octylacrylamide/acrylate/butylaminoethyl methacrylate copolymer), Advantage™ HC 37 (ISP; terpolymer of vinylcaprolactam/vinylpyrrolidone/dimethylaminoethyl methacrylate), Advantage grades (ISP), Acudyne 258 (Rohm & Haas; acrylate/hydroxy ester acrylate copolymer), Luviset™ P.U.R. (BASF, polyurethane-1), Luviflex™ Silk (BASF, PEG/PPG-25/25 dimethicone/acrylates copolymer), Eastman™ AQ48 (Eastman), Styleze 2000 (ISP; VP/acrylates/lauryl methacrylate copolymer), Styleze CC-10 (ISP; VP/DMAPA acrylates copolymer), Styleze W-20 (ISP), Fixomer A-30 (Ondeo Nalco; methacrylic acid/sodium acrylamidomethylpropanesulfonate copolymer), Fixate G-100 (Noveon; AMP-acrylates/allyl methacrylate copolymer).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCl name Polyquaternium, e.g. copolymers of vinylpyrrolidone/N-vinylimidazolium salts (Luviquat™ FC, Luviquat™ HM, Luviquat™ MS, Luviquat™ Care, INCl: Polyquaternium-16, Polyquaternium-44), copolymers of N-vinylpyrrolidone/dimethylaminoethyl methacrylate, quaternized with diethyl sulfate (Luviquat™ PQ 11, INCl: Polyquaternium-11), copolymers of N-vinylcaprolactam/N-vinylpyrrolidone/N-vinylimidazolium salts (Luviquat™ Hold, INCl: Polyquaternium-46); copolymers of acrylamide and dimethyldiallylammonium chloride (Polyquaternium-7), cationic cellulose derivatives (Polyquaternium-4, -10), cationic starch derivatives (INCl: Starch Hydroxypropytrimonium Chloride, Corn Starch Modified), cationic guar derivatives (INCl: Hydroxypropyl Guar Hydroxypropyltrimonium Chloride), cationic sunflower oil derivatives (INCl: Sunflowerseedamidopropyl Hydroxyethyldimonium Chloride), copolymers of acrylic acid, acrylamide and methacrylamidopropyltrimonium chloride (INCl: Polyquaternium-53), Polyquaternium-32, Polyquaternium-28 and others.

Further suitable hair cosmetic polymers are also neutral polymers, such as polyvinylpyrrolidones, copolymers of N-vinylpyrrolidone and vinyl acetate and/or vinyl propionate, copolymers of N-vinylpyrrolidone/dimethylaminopropylacrylamide or -methacrylamide, copolymers of N-vinylpyrrolidone and alkyl acrylate or methacrylate monomers with alkyl chains from C1 to C18, graft copolymers of polyvinyl alcohol onto polyalkylene glycols, such as, for example, Kollicoat IR (BASF), graft copolymers of other vinyl monomers onto polyalkylene glycols, polysiloxanes, polyvinylcaprolactam and copolymers containing N-vinylpyrrolidone, polyethylenimines and salts thereof, polyvinylamines and salts thereof, cellulose derivatives, chitosan, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins, fluorinated alkylsilicones, dimethicones, dimethicone derivatives or dimethicone copolyols (CTFA) and aminofunctional silicone compounds, such as amodimethicones (CTFA).

The polymers according to the invention are particularly suitable as setting agents in hairstyling preparations, in particular hair sprays (aerosol sprays and pump sprays without propellant gas) and hair mousses (aerosol mousses and pump mousses without propellant gas).

In a preferred embodiment, these preparations comprise

a) 0.1 to 10% by weight of the polymeric product according to the invention

b) 20 to 99.9% by weight of water and/or alcohol

c) 0 to 70% by weight of a propellant

d) 0 to 20% by weight of further constituents

Propellants are the propellants customarily used for hair sprays or aerosol mousses. Preference is given to mixtures of propane/butane, pentane, dimethyl ether, 1,1-difluoroethane (HFC-152 a), carbon dioxide, nitrogen or compressed air.

A formulation for aerosol hair mousses preferred according to the invention comprises

a) 0.1 to 10% by weight of the polymeric product according to the invention

b) 55 to 99.8% by weight of water and/or alcohol

c) 5 to 20% by weight of a propellant

d) 0.1 to 5% by weight of an emulsifier

e) 0 to 10% by weight of further constituents

The emulsifiers used may be all emulsifiers customarily used in hair mousses. Suitable emulsifiers may be nonionic, cationic or anionic or amphoteric.

Examples of nonionic emulsifiers (INCl nomenclature) are laureths, e.g. laureth-4; ceteths, e.g. cetheth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are or bromide (INCl Cetrimonium chloride or bromide), hydroxyethylcetyldimonium phosphate (INCl Quaternium-44), INCl Cocotrimonium methosulfate, INCl Quaternium-52, Quaternium-1 to x (INCl).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, alkylglycol alkoxylates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units in the molecule.

A preparation suitable according to the invention for styling gels may, for example, have the following composition:

a) 0.1 to 10% by weight of the polymeric products according to the invention

b) 60 to 99.85% by weight of water and/or alcohol

c) 0.05 to 10% by weight of a gel former

d) 0 to 20% by weight of further constituents

Gel formers which can be used are all gel formers customary in cosmetics. These include slightly crosslinked polyacrylic acid, for example Carborner (INCI), cellulose derivatives, e.g. hydroxypropylcellulose, hydroxyethylcellulose, cationically modified celluloses, polysaccharides, e.g. xanthan gum, caprylic/capric triglycerides, Sodium Acrylates Copolymer, Polyquaternium-32 (and) Paraffinum Liquidum (INCl), Sodium Acrylates Copolymer (and) Paraffinum Liquidum (and) PPG-1 trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycole Dicaprate Dicaprylate (and) PPG-1 Trideceth-6, Polyquaternium-7, Polyquaternium-44.

The polymeric products according to the invention can be used in cosmetic preparations as conditioners. Examples are rinse-off and leave-on conditioner preparations.

The polymeric products according to the invention can also be used in shampoo formulations as setting and/or conditioning agents. Suitable as conditioning agents are, in particular, polymers with a cationic charge. Preferred shampoo formulations comprise

a) 0.05 to 10% by weight of the polymeric products according to the invention

b) 25 to 94.95% by weight of water

c) 5-50% by weight of surfactants

c) 0-5% by weight of a further conditioning agent

d) 0-10% by weight of further cosmetic constituents

In the shampoo formulations, all anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkylglycol alkoxylates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable compounds are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which can be linear or branched, containing ethylene oxide and/or propylene oxide. The amount of alkylene oxide is about 6 to 60 mol per mole of alcohol. Also suitable are alkylamine oxides, mono- or dialkyl alkanolamides, fatty acid esters of polyethylene glycols, alkyl polyglycosides or sorbitan ether esters.

Furthermore, the shampoo formulations can comprise customary cationic surfactants, such as, for example, quaternary ammonium compounds, for example cetyltrimethylammonium chloride or bromide (INCl Cetrimonium chloride or bromide), hydroxyethylcetyldimonium phosphate (INCl Quaternium-44), INCl Cocotrimonium methosulfate, INCl Quaternium-52.

In the shampoo formulations, customary conditioners can be used in combination with the polymers according to the invention to achieve certain effects. These include, for example, cationic polymers with the INCl name Polyquaternium, e.g. copolymers of vinylpyrrolidone/N-vinylimidazolium salts (Luviquat™ FC, Luviquat™ HM, Luviquat™ MS, Luviquat™ Care, INCl: Polyquaternium-16, Polyquaternium44), copolymers of N-vinylpyrrolidone/dimethylaminoethyl methacrylate, quaternized with diethyl sulfate (Luviquat™ PQ 11, INCl: Polyquaternium-11), copolymers of N-vinylcaprolactam/N-vinylpyrrolidone/N-vinylimidazolium salts (Luviquat™ Hold, INCl: Polyquaternium-46); copolymers of acrylamide and dimethyldiallylammonium chloride (Polyquaternium-7), cationic cellulose derivatives (Polyquaternium-4, -10). It is also possible to use cationic starch derivatives (INCl: Starch Hydroxypropyltrimonium Chloride, Corn Starch Modified), cationic guar derivatives (INCl: Hydroxypropyl Guar Hydroxypropyltrimonium Chloride), cationic sunflower oil derivatives (INCl: Sunflowerseedamidopropyl Hydroxyethyldimonium Chloride), copolymers of acrylic acid, acrylamide and methacrylamidopropyltrimonium chloride (INCl: Polyquaternium-53), Polyquaternium-32, Polyquaternium-28 and others. In addition, it is possible to use protein hydrolysates, and conditioning substances based on silicone compounds, for example polyalkylsiloxanes, polyarylsiloxanes, polyarylalkylsiloxanes, polyethersiloxanes or silicone resins. Further suitable silicone compounds are dimethicones, dimethicone derivatives or dimethicone copolyols (CTFA) and aminofunctional silicone compounds such as amodimethicones (CTFA).

### EXAMPLES

### Preparation Example

### Example 1

a) Preparation of Polymer (A)

The initial charge was heated under nitrogen gassing to 78° C. and left for 15 minutes at 78° C. Then, feed 1 was metered in over the course of 2 hours, and feed 2 was metered in over the course of 2.5 hours. The mixture was after-polymerized for 2 hours and then feed 3 was metered in over the course of 15 minutes.

Initial Charge:

250 g of ethanol, cosm.

63 g of Pluriol A 11 R

100 g of feed 1

7.5 g of feed 2

Feed 1:

120 g of ethanol, cosm.

562 g of tert-butyl acrylate

188 g of methacrylic acid

Feed 2:

200 g of ethanol, cosm.

3 g of tert-butyl perpivalate (75% strength)

Feed 3:

240 g of ethanol, cosm.

3 g of tert-butyl perpivalate (75% strength)

The resulting polymer A had a solids content of 51.3%, a K value (1% in ethanol) of 39.1 and an acid number (mg of KOH/g) of 80.1.

b) Mixing of (A) with Silicone (B)

In a 2 l four-necked flask, 243.7 g of the polymer obtained as in a) and 13.84 g of polyalkoxylated silicone (Belsil DMC 6031, Wacker) were stirred at 78° C. for one hour under nitrogen gassing.

The resulting product had a solids content of 55.9%, a K value (1% in ethanol) of 37.1 and an acid number (mg of KOH/g) of 81.6.

### Comparative Example 1

For Comparative Example 1, the procedure was analogous to Example 1 (polymer A and silicone B). However, the two components were stirred at room temperature (20° C.) for 1 hour.

Performance Properties

a) Measurement of the Stiffness (Flexural Strength)

To measure the flexural strength, 3.0% strength by weight solutions of the polymeric products according to Example 1 and of Comparative Example 1 were prepared. The flexural strength measurement was carried out on 5 to 10 hair tresses (each about 3 g and 24 cm in length) at 20° C. and a relative humidity of 65%. The weighed dry hair tresses were dipped into the 3% strength polymer solution, uniform distribution being ensured by immersing and removing the tresses three times. The excess film former solution was stripped off between the thumb and index finger and the tresses of hair were then carefully squeezed out by pressing between filter papers. The hair tresses were then shaped by hand such that they had a round cross section. They were dried overnight in a climatically controlled room at 20° C. and a relative humidity of 65%.

The tests were carried out in a climatically controlled room at 20° C. and a relative humidity of 65% using a tensile/compressive testing device. The tress of hair was placed symmetrically on two cylindrical rolls of the sample holder. Then, the hair tresses were bent 40 mm exactly in the middle from above using a rounded punch (breakage of the polymer film). The force required for this was measured using a load cell (50 N) and given in newtons.

b) Measuring the Curl Retention

To measure the curl retention, 1.8% strength by weight solutions of the polymeric product according to Example 1 and of Comparative Example 1 were prepared. The curl retention measurement was carried out as follows: the washed, dry hair tresses were placed in 50% strength ethanol (ethanol abs./water dist. 1:1) at about 40° C. for 15 minutes. The thumb and index finger were used to press out the excess liquid and the hair tresses were wound around a Plexiglas tube. The hair tresses were then dried overnight at 65 to 70° C. After 15 minutes at room temperature, the hair was unwound. About 5 g of hair spray was sprayed on (from a distance of about 20 cm), during which the curl was rotated. Subsequently, it was dried in the lying position for 1 hour at room temperature.

The curl was hung up by one end and the curl length (Lo) was measured. The curl was placed into a climatically controlled chamber (25° C., relative humidity 90%) and its length (Lt) after 15, 30, 60 and 90 minutes, and also after 2, 3, 4, 5 and 24 hours was measured. The test was carried out on at least 5 hair tresses.

$$Curl Retention in \% = \frac{L - Lt}{L - Lo} * 100$$

L=Length of the hair (15.5 cm)

Lo=Length of the hair after drying

Lt=Length of the hair following climatically controlled treatment

Table 1 gives the values of the flexural strength and curl retention of Example 1 and Comparative Example 1.

TABLE 1
Flexural strength in cN (3% by weight W.S. in ethanol abs.; 20° C.
and relative humidity 65%) and curl retention (%)

| | Flexural | Curl |
|---|---|---|
| | strength [cN] | retention (%) |
| Comparative Example 1 | 79 | 78 |
| Example 1 | 126 | 76 |

As the table shows, the flexural strength of the polymeric product according to the invention is significantly greater than that of the comparative example whilst retaining the curl retention properties.

Formulation Examples

| | Mixing ratio | Mixing ratio | Mixing ratio |
|---|---|---|---|
| Polymers | 8:2 | 1:1 | 2:8 |
| Polymeric | 6.40% polymeric product | 4.0% polymeric product | 1.6% polymeric product |
| product as in | as in Example 1 | as in | as in |
| Example 1/ | 1.60% Luviskol | Example 1 | Example 1 |
| Luviskol | VA37E ™ | 4.0% Luviskol | 6.4% Luviskol |
| VA37 ™ | 0.74% AMP (2-amino-2- | VA37E ™ | VA37E ™ |
| | methylpropanol) | 0.46% AMP | 0.19% AMP |
| | 51.26% ethanol | 51.54% ethanol | 51.81% ethanol |
| | 40.00% DME (dimethyl | 40.0% DME | 40.0% DME |
| | ether) | | |
| Polymeric | 6.4% polymeric product | 4.0% polymeric product | 1.6% polymeric product |
| product as in | as in Example 1 | as in | as in |
| Example 1/ | 0.8% Luviskol K30 ™ | Example 1 | Example 1 |
| Luviskol | 0.74% AMP | 2.0% Luviskol K30 ™ | 3.2% Luviskol K30 ™ |
| K30 ™ | 52.06% ethanol | 0.46% AMP | 0.19% AMP |
| | 40.0% DME | 53.54% ethanol | 55.01% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 2.0% Luviskol Plus ™ | Example 1 | Example 1 |
| Luviskol | 0.74% AMP | 5.0% Luviskol Plus ™ T | 8.0% Luviskol Plus ™ |
| Plus ™ | 50.86% ethanol | 0.46% AMP | 0.19% AMP |
| | 40.0% DME | 50.54% ethanol | 50.21% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 2.67% Luviset P.U.R ™ | Example 1 | Example 1 |
| Luviset | 0.73% AMP | 6.67% Luviset | 10.67% Luviset |
| P.U.R. | 50.20% ethanol | P.U.R ™ | P.U.R ™ |
| | 40.0% DME | 0.46% AMP | 0.19% AMP |
| | | 48.87% ethanol | 47.54% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 0.8% Ultrahold 8 ™ | Example 1 | Example 1 |
| Ultrahold 8 | 0.82% AMP | 2.0% Ultrahold 8 ™ | 3.2% Ultrahold 8 ™ |
| | 51.98% ethanol | 0.66% AMP | 0.51% AMP |
| | 40.0% DME | 53.34% ethanol | 54.69% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 0.8% Ultrahold | Example 1 | Example 1 |
| Ultrahold | StrongT | 2.0% Ultrahold | 3.2% Ultrahold |
| Strong | 0.84% AMP | StrongT | StrongT |
| | 51.96% ethanol | 0.71% AMP | 0.59% AMP |
| | 40.0% DME | 53.29% ethanol | 54.61% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 0.8% Luviset CA66 ™ | Example 1 | Example 1 |
| Luviset CA66 | 0.82% AMP | 2.0% Luviset CA66 ™ | 3.2% Luviset CA66 ™ |
| | 51.98% ethanol | 0.69% AMP | 0.53% AMP |
| | 40.0% DME | 53.31% ethanol | 54.67% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 0.8% Luviset CAN ™ | Example 1 | Example 1 |
| Luviset CAN | 0.82% AMP | 2.0% Luviset CAN ™ | 3.2% Luviset CAN ™ |
| | 51.98% ethanol | 0.70% AMP | 0.54% AMP |
| | 40.0% DME | 53.3% ethanol | 54.66% ethanol |
| | | 40.0% DME | 40.0% DME |
| Polymeric | 6.4% polymeric product | 4.0% polymeric product | 1.6% polymeric product |
| product as in | as in Example 1 | as in | as in |
| Example 1/ | 0.8% Amphomer ™ | Example 1 | Example 1 |
| Amphomer | 0.88% AMP | 2.0% Amphomer ™ | 3.2% Amphomer ™ |
| | 51.92% ethanol | 0.83% AMP | 0.77% AMP |
| | 40.0% DME | 52.87% ethanol | 54.43% ethanol |
| | | 40.0% DME | 40.0% DME |

Hair sprays containing propane/butane 3.5 bar or propane/
butane 3.5 bar with added DME

| | Mixing ratio | Mixing ratio | Mixing ratio |
|---|---|---|---|
| Polymers | 8:2 | 1:1 | 2:8 |
| Polymeric product | 6.4% polymeric product | 4.0% polymeric product | 1.6% polymeric product |
| as in Example | as in | as in | as in |
| 1/Luviskol | Example 1 | Example | Example 1 |
| VA37 | 1.6% Luviskol | 14.0% Luviskol | 6.4% Luviskol |
| | VA37E ™ | VA37E ™ | VA37E ™ |
| | 0.74% AMP | 0.46% AMP | 0.19% AMP |
| | 51.26% ethanol | 51.54% ethanol | 51.81% ethanol |
| | 10.0% Pr/Bu 3.5 (propane/ | 10.0% Pr/Bu 3.5 | 10.0% Pr/Bu 3.5 |
| | butane | 30.0% DME | 30.0% DME |
| | 3.5 bar) | | |
| | 30.0% DME | | |
| Polymeric | 6.4% polymeric product | 4.0% polymeric product | 1.6% polymeric product |
| product as in | as in | as in | as in |
| Example 1/ | Example 1 | Example 1 | Example 1 |
| Luviskol K30 | 0.8% Luviskol K30 ™ | 2.0% Luviskol K30 ™ | 3.2% Luviskol K30T |
| | 0.74% AMP | 0.46% AMP | 0.19% AMP |
| | 52.06% ethanol | 53.54% ethanol | 55.01% ethanol |
| | 40.0% Pr/Bu 3.5 | 40.0% Pr/Bu 3.5 | 40.0% Pr/Bu 3.5 |
| Polymeric | 6.4% polymeric product | 4.0% polymeric product | 1.6% polymeric product |
| product as in | as in | as in | as in |
| Example 1/ | Example 1 | Example 1 | Example 1 |
| Luviskol Plus | 2.0% Luviskol PlusT | 5.0% Luviskol PlusT | 8.0% Luviskol PlusT |
| | 0.74% AMP | 0.46% AMP | 0.19% AMP |
| | 50.86% ethanol | 50.54% ethanol | 50.21% ethanol |
| | 40.0% Pr/Bu 3.5 | 40.0% Pr/Bu 3.5 | 40.0% Pr/Bu 3.5 |
| Polymeric | Not possible | 4.0% polymeric product | 1.6% polymeric product |
| product as in | | as in | as in Example |
| Example 1/ | | Example 1 | 110.67% Luviset |
| Luviset P.U.R. | | 6.67% Luviset | P.U.R ™ |
| | | P.U.R ™ | 0.19% AMP |
| | | 0.46% AMP | 47.54% ethanol |
| | | 48.87% ethanol | 15% Pr/Bu 3.5 |
| | | 10% Pr/Bu 3.5 | 25.0% DME |
| | | 30.0% DME | |
| Polymeric | 6.4% polymeric product | 4.0% polymeric product | 1.6% polymeric product |
| product as in | as in | as in | as in |
| Example 1/ | Example 1 | Example 1 | Example 1 |
| Luvimer 100P | 0.8% Luvimer 100P ™ | 2.0% Luvimer 100P ™ | 3.2% Luvimer 100P ™ |
| | 0.93% AMP | 0.94% AMP | 0.95% AMP |
| | 51.87% ethanol | 53.06% ethanol | 54.25% ethanol |
| | 40.0% Pr/Bu 3.5 | 40.0% Pr/Bu 3.5 | 40.0% Pr/Bu 3.5 |
| Polymeric | 6.4% polymeric product | 4.0% polymeric product | 1.6% polymeric product |
| product as in | as in | as in | as in |
| Example 1/ | Example 1 | Example 1 | Example 1 |
| Ultrahold 8 | 0.8% Ultrahold 8 ™ | 2.0% Ultrahold 8 ™ | 3.2% Ultrahold 8 ™ |
| | 0.82% AMP | 0.66% AMP | 0.51% AMP |
| | 51.98% ethanol | 53.34% ethanol | 54.69% ethanol |
| | 40.0% Pr/Bu 3.5 | 40.0% Pr/Bu 3.5 | 40.0% Pr/Bu 3.5 |
| Polymeric | 6.4% polymeric product | 4.0% polymeric product | 1.6% polymeric product |
| product as in | as in | as in | as in |
| Example 1/ | Example | Example 1 | Example 1 |
| Ultrahold | 10.8% Ultrahold | 2.0% Ultrahold | 3.2% Ultrahold |
| Strong | Strong ™ | Strong ™ | Strong ™ |
| | 0.84% AMP | 0.71% AMP | 0.59% AMP |
| | 51.96% ethanol | 53.29% ethanol | 54.61% ethanol |
| | 40.0% Pr/Bu 3.5 | 40.0% Pr/Bu 3.5 | 40.0% Pr/Bu 3.5 |
| Polymeric | 6.4% polymeric product | 4.0% polymeric product | 1.6% polymeric product |
| product as in | as in | as in | as in |
| Example 1/ | Example 1 | Example 1 | Example |
| Luviset CA66 | 0.8% Luviset CA66 ™ | 2.0% Luviset CA66 ™ | 13.2% Luviset |
| | 0.82% AMP | 0.69% AMP | CA66 ™ |
| | 51.98% ethanol | 53.31% ethanol | 0.53% AMP |
| | 10.0% Pr/Bu 3.5 | 10.0% Pr/Bu 3.5 | 54.67% ethanol |
| | 30.0% DME | 30.0% DME | 10.0% Pr/Bu 3.5 |
| | | | 30.0% DME |
| Polymeric | 6.4% polymeric product | 4.0% polymeric product | 1.6% polymeric product |
| product as in | as in | as in | as in |
| Example 1/ | Example 1 | Example 1 | Example 1 |
| Luviset ™ CAN | 0.8% Luviset CAN ™ | 2.0% Luviset CAN ™ | 3.2% Luviset CAN ™ |
| | 0.82% AMP | 0.70% AMP | 0.54% AMP |
| | 51.98% ethanol | 53.3% ethanol | 54.66% ethanol |
| | 20.0% Pr/Bu 3.5 | 20.0% Pr/Bu 3.5 | 20.0% Pr/Bu 3.5 |
| | 20.0% DME | 20.0% DME | 20.0% DME |
| Polymeric | 6.4% polymeric product | 4.0% polymeric product | 1.6% polymeric product |
| product as in | as in | as in | as in |
| Example 1/ | Example 1 | Example 1 | Example 1 |
| Amphomer ™ | 0.8% Amphomer ™ | 2.0% Amphomer ™ | 3.2% Amphomer ™ |
| | 0.88% AMP | 0.83% AMP | 0.77% AMP |
| | 51.92% ethanol | 52.87% ethanol | 54.43% ethanol |
| | 40.0% Pr/Bu 3.5 | 40.0% Pr/Bu 3.5 | 40.0% Pr/Bu 3.5 |
| Polymeric | 6.4% polymeric product | 4.0% polymeric product | 1.6% polymeric product |
| product as in | as in | as in | as in |
| Example 1/ | Example 1 | Example 1 | Example 1 |
| Luviflex Silk ™ | 0.8% Luviflex Silk ™ | 2.0% Luviflex Silk ™ | 3.2% Luviflex Silk ™ |
| | 0.88% AMP | 0.83% AMP | 0.77% AMP |
| | 51.92% ethanol | 52.87% ethanol | 54.43% ethanol |
| | 40.0% Pr/Bu 3.5 | 40.0% Pr/Bu 3.5 | 40.0% Pr/Bu 3.5 |

Pump sprays

| | Mixing ratio | Mixing ratio | Mixing ratio |
|---|---|---|---|
| Polymers | 8:2 | 1:1 | 2:8 |
| Polymeric | 10.85% polymeric | 6.74% polymeric | 2.69% polymeric |
| product as in | product as in | product as in | product as in |
| Example 1/ | Example 1 | Example 1 | Example 1 |
| Luviskol VA37 | 2.66% Luviskol | 6.66% Luviskol | 10.64% |
| | VA37E ™ | VA37E ™ | Luviskol ™ VA37E |
| | 1.27% AMP | 0.79% AMP | 0.32% AMP |
| | 85.22% ethanol | 85.81% ethanol | 86.35% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviskol K30 | 1.33% Luviskol K30 ™ | 3.33% Luviskol K30 ™ | 5.32% Luviskol K30 ™ |
| | 1.27% AMP | 0.79% AMP | 0.32% AMP |
| | 86.55% ethanol | 84.14% ethanol | 83.69% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviskol Plus | 3.33% Luviskol Plus ™ | 8.33% Luviskol Plus ™ | 13.3% Luviskol Plus ™ |
| | 1.27% AMP | 0.79% AMP | 0.32% AMP |
| | 84.55% ethanol | 89.14% ethanol | 90.21% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviset P.U.R. | 4.43% Luviset | 11.1% Luviset | 17.73% Luviset |
| | P.U.R ™ | P.U.R ™ | P.U.R ™ |
| | 1.27% AMP | 0.79% AMP | 0.32% AMP |
| | 83.45% ethanol | 81.37% ethanol | 79.26% ethanol |
| Polymeric | 10.85 polymeric product | 6.74% polymeric | 2.69% polymeric |
| product as in | as in | product as in | product as in |
| Example 1/ | Example 1 | Example 1 | Example 1 |
| Luvimer 100p | 1.33% Luvimer | 3.33% Luvimer | 5.32% Luvimer |
| | 100P ™ | 100P ™ | 100P ™ |
| | 1.55% AMP | 1.56% AMP | 1.58% AMP |
| | 86.27% ethanol | 88.37% ethanol | 90.41% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Ultrahold 8 | 1.33% Ultrahold 8 ™ | 3.33% Ultrahold 8 ™ | 5.32% Ultrahold 8 ™ |
| | 1.36% AMP | 1.1% AMP | 0.85% AMP |
| | 86.46% ethanol | 88.83% ethanol | 91.14% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Ultrahold | 1.33% Ultrahold | 3.33% Ultrahold | 5.32% Ultrahold |
| Strong | Strong ™ | Strong ™ | Strong ™ |
| | 1.4% AMP | 1.18% AMP | 0.98% AMP |
| | 86.42% ethanol | 88.75% ethanol | 91.01% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviset CA66 | 1.33% Luviset | 3.33% Luviset | 5.32% Luviset |
| | CA66 ™ | CA66 ™ | CA66 ™ |
| | 1.36% AMP | 1.15% AMP | 0.88% AMP |
| | 86.46% ethanol | 88.78% ethanol | 91.11% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviset CAN | 1.33% Luviset CAN ™ | 3.33% Luviset CAN ™ | 5.32% Luviset CAN ™ |
| | 1.37% AMP | 1.17% AMP | 0.9% AMP |
| | 86.45% ethanol | 88.76% ethanol | 91.09% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Amphomer | 1.33% Amphomer ™ | 3.33% Amphomer ™ | 3.33% Amphomer ™ |
| | 1.47% AMP | 1.38% AMP | 1.28% AMP |
| | 86.35% ethanol | 88.55% ethanol | 92.7% ethanol |
| Polymeric | 10.85% polymeric | 6.74% polymeric | 2.69% polymeric |
| product as in | product as in | product as in | product as in |
| Example 1/ | Example 1 | Example 1 | Example 1 |
| Luviflex Silk ™ | 1.33% Luviflex Silk ™ | 3.33% Luviflex Silk ™ | 3.33% Luviflex Silk ™ |
| | 1.47% AMP | 1.38% AMP | 1.28% AMP |
| | 86.35% ethanol | 88.55% ethanol | 92.7% ethanol |

Hair spray formulation based on dimethyl ether

| | |
|---|---|
| 1.00% by weight | Luviskol K30 ™ (BASF) |
| 2.92% by weight | polymeric product as in Example 1 |
| 0.92% by weight | 2-amino-2-methylpropanol |
| 0.10% by weight | diisobutyl adipate (Ex. Crodanol DiBA from Croda |
| | Oleochemicals) |
| 0.05% by weight | isodecane |
| 0.10% by weight | perfume oil |
| 0.05% by weight | D-Panthenol USPT (BASF) |
| 14.78% by weight | water, demineralized |
| 36.08% by weight | ethanol |
| 40.00% by weight | dimethyl ether |

Hair spray formulations based on isobutane and n-pentane

| | | |
|---|---|---|
| A) | 6.80% | polymeric product as in Example 1 |
| | 0.79% | 2-amino-2-methylpropanol |
| | 14.20% | n-pentane |
| | 2.40% | n-butane |
| | 35.90% | isobutane |
| | 39.91% | ethanol |
| B) | 3.00% | Ultrahold Strong ™ (BASF) |
| | 1.00% | polymeric product as in Example 1 |
| | 0.48% | 2-amino-2-methylpropanol |
| | 0.03% | DOW Corning 190 ™ (Dow Corning) |
| | 14.20% | n-pentane |
| | 2.40% | n-butane |
| | 35.90% | isobutane |
| | 42.99% | ethanol |

Shine spray

| | |
|---|---|
| 2.00% | Luviset CA66 ™ (BASF) |
| 2.00% | polymeric product as in Example 1 |
| 0.46% | 2-amino-2-methylpropanol |
| 1.00% | DOW Corning 556 (Dow Corning) |
| 0.10% | niacinamide |
| 0.20% | D-Panthenol USP ™ (BASF) |
| 14.20% | n-pentane |
| 35.90% | n-butane |
| 44.14% | ethanol |

Hair spay VOC 80 with HFC 152A

| | |
|---|---|
| 2.00% | Luviset CA66 ™(BASF) |
| 4.80% | polymeric product as in Example 1 |
| 0.79% | 2-amino-2-methylpropanol |
| 56.60% | ethanol |
| 15.81% | propellant 152a (Ex. Dymel 152a from DuPont) |
| 20.00% | dimethyl ether |

Hair spray VOC 55 with vitamins

| | |
|---|---|
| 4.80% | polymeric product as in Example 1 |
| 3.33% | Luviset P.U.R. ™ |
| 0.57% | 2-amino-2-methylpropanol |
| 0.10% | niacinamide (Hoffmann-La Roche) |
| 0.10% | Panthenol USP ™ (BASF) |
| 38.83% | water, demineralized |
| 12.27% | ethanol |
| 40.00% | dimethyl ether |

Sunscreen pump spray for hair

| | |
|---|---|
| 2.00% | polymeric product as in Example 1 |
| 0.23% | 2-amino-2-methylpropanol |
| 2.00% | Uvinul MS 40 ™ (BASF) (benzophenone-4) |
| 95.77% | ethanol |

Hair repair

| | |
|---|---|
| 1.00% | Luviskol K30 ™ (BASF) |
| 4.00% | polymeric product as in Example 1 |
| 0.48% | 2-amino-2-methylpropanol |
| 0.20% | hydrolyzed wheat protein (Ex. Cropesol WT from Croda, Inc.) |
| 0.50% | D-Panthenol USP ™ (BASF) |
| 5.00% | 1,2-Propylene Glycol USP ™ (BASF) |
| 10.00% | ethanol |
| 78.82% | water, demineralized |

Shining gel for hair with UV protection

| | | |
|---|---|---|
| Phase A | 0.80% | Carbopol 2001 ETD ™ (Goodrich) |
| | 33.84% | water, demineralized |
| Phase B | 5.00% | Abil 200 (Goldschmidt) |
| | 3.00% | Karion FP (Merck KGaA) |
| | 3.00% | 1,2-Propylene glycol USPT (BASF) |
| | 1.00% | Cremophor RH40T (BASF) |
| | q.s. | preservative |
| Phase C | 50.00% | water, demineralized |
| | 0.50% | Uvinul P25 ™ (BASF) (PEG-25PABA) |
| | 2.00% | polymeric product as in Example 1 |
| | 0.23% | 2-amino-2-methylpropanol |
| Phase D | 0.63% | 2-amino-2-methylpropanol |

Mascara

| | | |
|---|---|---|
| Phase A | 1.50% | Cremophor A6 ™ (BASF) |
| | 1.50% | Cremophor A25 ™ (BASF) |
| | 2.00% | stearic acid (Ex. Emersol 120 ™ from |
| | | Henkel) |
| | 3.00% | Imwitor 960 K ™ (Hüls AG) |
| | 3.00% | Softisan 100 ™ (Hüls AG) |
| | 1.50% | Luvigel EM ™ (BASF) |
| | 10.00% | Dow Corning 345 ™ (Dow Corning) |
| Phase B | 4.00% | polymeric product as in Example 1 |
| | 0.46% | 2-amino-2-methylpropanol |
| | 0.30% | Germal 115 ™ (Sutton) |
| | 72.24% | water, demineralized |
| Phase C | 0.50% | phenoxyethanol (Ex. Phenoxetol ™ from |
| | | Nipa-Hardwicke) |

Shampoo formulation

| | |
|---|---|
| 1.50% | polymeric product as in Example 1 |
| 0.17% | 2-aminomethylpropanol |
| 0.50% | Luviskol K30 ™ (BASF) |
| 10.00% | Tego-Betaine L7 |
| 40.00% | Texapon NSO |
| 0.10% | Euxyl K100 |
| 2.00% | NaCl |
| 45.73% | water |

Shampoo formulation with Luviquat Care ™

| | |
|---|---|
| 1.80% | polymeric product as in Example 1 |
| 0.21% | 2-aminomethylpropanol |
| 0.20% | Luviskol K30 ™ (BASF) |
| 7.70% | Luviquat Care ™ (BASF) |
| 10.00% | Tego-Betaine L7 |
| 40.00% | Texapon NSO |
| 0.10% | Euxyl K 100 |
| 2.00% | NaCl |
| 37.99% | water |

Clear lacquer

| | |
|---|---|
| 15.0% | nitrocellulose |
| 7.0% | polymeric product as in Example 1 |
| 2.4% | camphor |
| 4.5% | palatinol A |
| 7.0% | isopropanol |
| 8.0% | methyl acetate |
| 8.0% | ethyl acetate |
| 14.0% | ispropyl acetate |
| 34.1% | butyl acetate |

Clear lacquer without nitrocellulose

| | |
|---|---|
| 23.0% | polymeric product as in Example 1 |
| 7.0% | Ketjenflex MH |
| 2.4% | camphor |
| 4.5% | palatinol A |
| 8.0% | methyl acetate |
| 8.0% | ethyl acetate |
| 14.0% | isopropyl acetate |
| 33.1% | butyl acetate |

VOC 55 hair spray

| | |
|---|---|
| 21.50% | water |
| 35.00% | alcohol SD 40-B |
| 0.95% | aminomethylpropanol |
| 8.00% | polymeric product as in Example 1 |
| 0.20% | D,L-panthenol |
| 0.10% | Uvinul ™ MC 80 (octyl methoxycinnamate) |
| 0.10% | Masil ™ SF 19 (dimethicone copolyol) |
| 15.00% | dimethyl ether |
| 20.00% | hydrofluorocarbon 152a |

VOC 55 hair spray

| | |
|---|---|
| 34.10% | water |
| 52.00% | alcohol SD 40-B |
| 0.50% | aminomethylpropanol |
| 9.00% | Luviset ™ P.U.R. polyurethane-1) |
| 4.00% | polymeric product as in Example 1 |
| 2.00% | D,L-panthenol |
| 0.10% | Uvinul ™ MC 80 (octyl methoxycinnamate) |
| 0.10% | Masil ™ SF 19 (dimethicone copolyol) |

Unless expressly mentioned otherwise, all of the percentages given in the examples are percentages by weight.

1. A process for the preparation of polymeric products, which comprises
i) polymerizing ethylenically unsaturated monomers (A-1) in the presence of unsaturated polyalkylene glycols (A-2)
ii) mixing the resulting polymer with silicones (B) at a temperature of greater than or equal to 40° C., in particular greater than or equal to 50° C.

2. The process according to claim 1, wherein the silicones (B) are chosen from the group consisting of (B-1) silicones with at least one quaternized or nonquaternized amine function, (B-2) silicone resins, (B-3) silicone rubbers, (B-4) polyalkoxylated silicones and/or silicone-containing polyurethanes (B-5).

3. A polymeric product obtainable by mixing
(A) polymers which are obtainable by free-radical polymerization of (A-1) ethylenically unsaturated monomers in the presence of (A-2) unsaturated polyalkylene glycols with
(B) silicones
at a temperature greater than or equal to 40° C., in particular greater than or equal to 50° C.

4. The polymeric product according to claim 3, wherein the ethylenically unsaturated monomers (A-1) and the unsaturated polyalkylene glycols (A-2) are polymerized in the ratio 50 to 99.9% by weight of (A-1) and 0.1 to 50% by weight of (A-2).

5. The polymeric product according to claim 3, wherein 99.5 to 70% by weight of polymer (A) and 0.5 to 30% by weight of silicones (B) are used.

6. The polymeric product according to claim 3, wherein the silicones are chosen from the group consisting of (B-1) silicones with at least one quaternized or nonquaternized amine function, (B-2) silicone resins, (B-3) silicone rubbers, (B-4) polyalkoxylated silicones and/or silicone-containing polyurethanes (B-5).

7. The polymeric product according to claim 3, wherein the monomer (A-1) used is at least one compound of the formula
X—C(O)CR⁷═CHR⁶
where
X is chosen from the group of radicals —OH, —OM, —OR⁸, NH₂, —NHR⁸, N(R⁸)₂
M is a cation chosen from the group consisting of: Na+, K+, Mg++, Ca++, Zn++, NH4+, alkylammonium, dialkylammonium, trialkylammonium and tetraalkylammonium;
the radicals R⁸ may be identical or different and are chosen from the group consisting of —H, C1-C40 linear or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.
R⁷ and R⁶, independently of one another, are chosen from the group consisting of: —H, C₁-C₈ linear or branched-chain alkyl chains, methoxy, ethoxy, 2-hydroxyethoxy, 2-methoxyethoxy and 2-ethoxyethyl.

8. The polymeric product according to claim 3, wherein the monomers (A-1) are chosen from the group consisting of acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate and n-butyl methacrylate.

9. The polymeric product according to claim 3, wherein the monomers (A-1) used are at least 2 compounds which are chosen from the group consisting of acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate and n-butyl methacrylate.

10. The polymeric product according to claim 3, wherein the unsaturated polyalkylene glycols (A-2) used are compounds of the formula
H₂C═CH—C_{c}H_{2c}—O—(C₂H₄O)ₐ(C₃H₆O)_{b}—R⁵
and/or
H₂C═CH—C_{c}H_{2c}—O—(C₄H₈O)ₐ—R⁵
in which
R⁵ is —H, —CH═CH₂, —CH₂)₅—CH═CH—(CH₂)ₜ—H, a straight-chain or branched alkyl group having 1 to 12 carbon atoms, a straight-chain or branched alkoxy group having 1 to 6 carbon atoms, a straight-chain or branched acyl group having 2 to 40 carbon atoms, —SO₃M, a C₁₋₆-aminoacyl group optionally substituted on the amino group, —NHCH₂CH₂COOM, —N(CH₂CH₂COOM)₂, an aminoalkyl group optionally substituted on the amino group and on the alkyl group, a C₂₋₃₀-carboxyacyl group, a phosphono group optionally substituted by one or two substituted aminoalkyl groups, —CO(CH₂)_{d}COOM, —COCHR⁷(CH₂)_{d}COOM, —NHCO(CH₂)_{d}OH, —NH₂Y or a phosphate group
the groups M, which are identical or different from one another, are hydrogen, Na, K, Li, NH₄ or an organic amine,
R⁷ is hydrogen or SO₃M,
d is in the range from 1 to 10,
s may be 0, 1 to 10
t may be 0, 1 to 10
a is in the range from 0 to 50,
b is in the range from 0 to 50,
a+b is greater than 0,
c is 0, 1, 2, 3 or4,
Y is a monovalent inorganic or organic anion.

11. A cosmetic preparation comprising a polymeric product according to claim 3.

12. The cosmetic preparation wherein said products sets according to claim 11 as a setting agent and/or conditioning agent.

13. A cosmetic preparation comprising
a) 0.05 to 20% by weight of the polymeric product according to claim 3
b) 20 to 99.95% by weight of water and/or alcohol
c) 0 to 79.5% by weight of further constituents
with the proviso that the amounts add up to 100%.

14. The polymeric product according to claim 4, wherein 99.5 to 70% by weight of polymer (A) and 0.5 to 30% by weight of silicones (B) are used.

15. The polymeric product according to claim 2, wherein the silicones are chosen from the group consisting of (B-1) silicones with at least one quaternized or nonquaternized amine function, (B-2) silicone resins, B-3) silicone rubbers, (B-4) polyalkoxylated silicones and/or silicone-containing polyurethanes (B-5).

16. The polymeric product according to claim 3, wherein the silicones are chosen from the group consisting of (B-1) silicones with at least one quaternized or nonquaternized amine function, (B-2) silicone resins, (B-3) silicone rubbers, (B4) polyalkoxylated silicones and/or silicone-containing polyurethanes (B-5).

17. The polymeric product according to claim 4, wherein the silicones are chosen from the group consisting of (B-1) silicones with at least one quaternized or nonquaternized amine function, (B-2) silicone resins, (B-3) silicone rubbers, (B-4) polyalkoxylated silicones and/or silicone-containing polyurethanes (B-5).

18. The polymeric product according to claim 5, wherein the silicones are chosen from the group consisting of (B-1) silicones with at least one quaternized or nonquaternized amine function, (B-2) silicone resins, (B-3) silicone rubbers, (B-4) polyalkoxylated silicones and/or silicone-containing polyurethanes (B-5).

19. The polymeric product according to claim 2, wherein the monomer (A-1) used is at least one compound of the formula
X—C(O)CR⁷═CHR⁶
where
X is chosen from the group of radicals —OH, —OM, —OR⁸, NH₂, —NHR⁸, N(R⁸)₂
M is a cation chosen from the group consisting of: Na+, K+, Mg++, Ca++, Zn++, NH4+, alkylammonium, dialkylammonium, trialkylammonium and tetraalkylammonium;
the radicals R⁸ may be identical or different and are chosen from the group consisting of —H, C1-C40 linear or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.
R⁷ and R⁶, independently of one another, are chosen from the group consisting of: —H, C₁-C₈ linear or branched-chain alkyl chains, methoxy, ethoxy, 2-hydroxyethoxy, 2-methoxyethoxy and 2-ethoxyethyl.

20. The polymeric product according to claim 3, wherein the monomer (A-1) used is at least one compound of the formula
X—C(O)CR⁷═CHR⁶
where
X is chosen from the group of radicals —OH, —OM, —R⁸, NH₂, —NHR⁸, N(R⁸)₂
M is a cation chosen from the group consisting of: Na+, K+, Mg++, Ca++, Zn++, NH4+, alkylammonium, dialkylammonium, trialkylammonium and tetraalkylammonium;
the radicals R⁸ may be identical or different and are chosen from the group consisting of —H, C1-C40 linear or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.
R⁷ and R⁶, independently of one another, are chosen from the group consisting of: —H, C₁-C₈ linear or branched-chain alkyl chains, methoxy, ethoxy, 2-hydroxyethoxy, 2-methoxyethoxy and 2-ethoxyethyl.
